(19) United States
(12) Patent Application Publication
(10) Pub. No.: US 2025/0259296 A1
(43) Pub. Date: Aug. 14, 2025

(54) SYSTEMS AND METHODS FOR INSPECTING A WORKSURFACE

(71) Applicant: 3M INNOVATIVE PROPERTIES COMPANY, St. Paul, MN (US)

(72) Inventors: Steven P. Floeder, Shoreview, MN (US); Jeffrey P. Adolf, Rochester, MN (US); Amy K. McNulty, Stillwater, MN (US); Jonathan B. Arthur, Hudson, WI (US); Caroline M. Ylitalo, Stillwater, MN (US); Jeffrey O. Emslander, Grant, MN (US); Julian D. Grant-Abban, Lake Elmo, MN (US); Alireza Ghaderi, Woodbury, MN (US); Rudy M. Lawler, Sioux Falls, SD (US); Robert A. Knutson, Lakeville, MN (US); David L. Hofeldt, Oakdale, MN (US)

(21) Appl. No.: 18/856,830

(22) PCT Filed: Apr. 13, 2023
(86) PCT No.: PCT/IB2023/053797

§ 371 Date: Oct. 14, 2024

Related U.S. Application Data
(60) Provisional application No. 63/363,063, filed on Apr. 15, 2022.

(51) Int. Cl. G06T 7/00; B25J 13/08; G01N 21/88; G06T 7/55

(52) U.S. Cl. CPC G06T 7/001; B25J 13/089; G01N 21/8806; G06T 7/55; G01N 2021/8822; G06T 2207/30108

(57) ABSTRACT

A method of evaluating a surface is presented that includes imaging the surface, with an imaging system. Imaging includes providing a camera of the imaging system proximate the surface. Imaging also includes causing the imaging system and the surface to move relative to each other, such that a distance between the imaging system and the surface is substantially maintained. Imaging also includes capturing image data of the surface. The image data is captured in a near dark field mode or a dark field image mode. The method also includes analyzing the image data and detecting a topography and/or appearance of the surface. The method also includes generating an evaluation regarding the surface based on the detected topography and/or surface appearance.

### BACKGROUND

Many industrial processes involve applying material to a worksurface. It is often important for the material to be evenly applied, without air bubbles, creases, or trapped debris. Techniques are desired for surface processing applications, including paint applications (e.g., primer sanding, clear coat defect removal, clear coat polishing, etc.), adhesive dispensing, film wrapping applications, or material removal systems are amenable to the use of abrasives and/or robotic inspection and repair.

### SUMMARY

A method of evaluating a surface is presented that includes imaging the surface, with an imaging system. Imaging includes providing a camera of the imaging system proximate the surface. Imaging also includes causing the imaging system and the surface to move relative to each other, such that a distance between the imaging system and the surface is substantially maintained. Imaging also includes capturing image data of the surface. The image data is captured in a near dark field mode or a dark field image mode. The method also includes analyzing the image data and detecting a topography and/or appearance of the surface. The method also includes generating an evaluation regarding the surface based on the detected topography and/or surface appearance.

### BRIEF DESCRIPTION OF THE DRAWINGS

In the drawings, which are not necessarily drawn to scale, like numerals may describe similar components in different views. The drawings illustrate generally, by way of example, but not by way of limitation, various embodiments discussed in the present document.

FIG. 1 illustrates a film wrapping process in which embodiments of the present invention are useful.

FIGS. 2A-2E illustrate operation of a line-scan array imaging system in accordance with embodiments herein.

FIGS. 3A-3B illustrate a line-scan array imaging system for a curved surface.

FIG. 4 illustrates a method of preparing and evaluating a surface after a material application in accordance with embodiments herein.

FIG. 5 illustrates a surface with a detected defect being addressed in one embodiment.

FIG. 6A-6F illustrates layers of a respirator that may be assembled and inspected using systems and methods herein.

FIG. 7A-7B illustrates a glass welding helmet that may benefit from systems and methods herein.

FIGS. 8A and 8B illustrate a prosthetic fitting that may benefit from systems and methods herein.

FIGS. 9A-9B illustrate tissue samples before and after a negative pressure treatment.

FIG. 10 illustrates a microreplicated surface that may be inspected using systems and methods herein.

FIG. 11 illustrates an adhesive dispensing operation that may benefit from systems and methods herein.

FIG. 12 illustrates an example topography detection system in accordance with embodiments herein.

FIG. 13 illustrates a surface inspection system in accordance with embodiments herein.

FIG. 14 illustrates a motive robot unit on which a topography mapping unit can be mounted in accordance with embodiments herein.

FIG. 15 illustrates a method of evaluating a surface in accordance with embodiments herein.

FIG. 16 is a defect inspection system architecture.

FIGS. 17-19 show examples of computing devices that can be used in embodiments shown in previous Figures.

FIGS. 20A-20B illustrate examples of surface processing and related calculations.

### DETAILED DESCRIPTION

Recent advancements in imaging technology and computational systems allowed for many surface preparation processes to happen at high speed. Adhesive can be prepared, dispensed and cured automatically. Often, defects are not located during a process, but only during a manual inspection once a process is complete. However, while some defects are within an acceptable tolerance, but others are not and result in a product being defective, either repairable or not. And many can be corrected by inspection during the process. A system is needed that can do rapid inspection so that processes are not slowed down, but the acceptable rate of products produced is improved.

As automated imaging of worksurfaces improves, it is equally possible to improve the ability to automatically process worksurfaces. It is desired to be able to detect and repair defects with as little manual intervention as possible. However, as discussed herein, many worksurface present challenges for imaging and quality control-including curvature, sharp surface features (corners, bends, grooves or other irregularities that deviate significantly from a flat surface at the area needing inspection. Similarly, such features make it difficult to obtain high fidelity images of a worksurface after a process has been done. It is important to quantify, classify and characterize defects in a processed surface. A more detailed understanding of the surface, and its physical location with respect to an imaging system is needed to obtain high fidelity images of the surface for quantitative evaluation.

Presented herein are embodiments of imaging systems and methods of use that may be beneficial to many processes. Imaging systems herein can be used with curved surfaces, flat surfaces, or irregular surfaces. In some embodiments, systems herein use a known expected topography—e.g. a retrieved CAD model or other known or expected information. In some embodiments, systems herein include distance sensors, or a distance sensor array, that helps to map topography of a worksurface.

As described herein, the term “worksurface” is used broadly to refer to a surface that undergoes a process that adds or removes material from the surface. Similarly, the term “material” is used broadly and may refer to, for example, a solid material (e.g. a wrapping or film layer), a liquid material (e.g. adhesive), a curable material, a 3D printed filament or structure, etc. Systems and methods herein are not intended to be construed as limited to the example use cases presented herein.

As used herein, the term “defect” refers to an area on a worksurface that interrupts the visual aesthetic, interrupts a pattern on the surface, an irregular surface, or debris trapped within, or on, the surface. Defects may be removeable post-imaging and pre-application of material, may be repairable post-application of material, or may cause a worksurface to be unsuitable for its intended application.

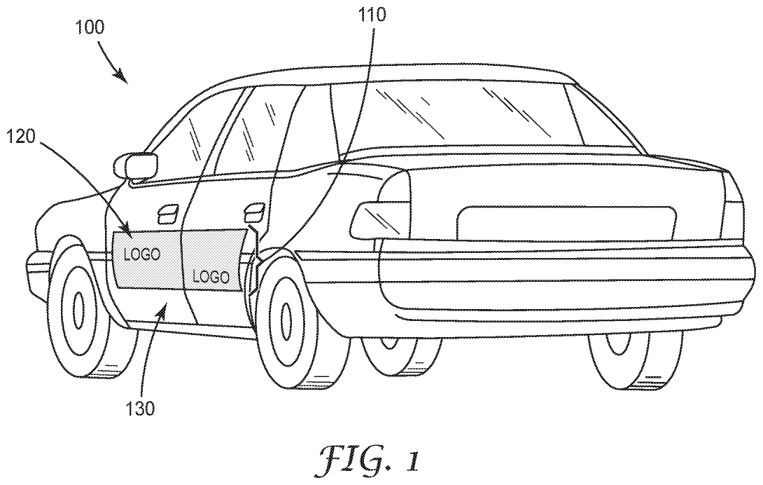

FIG. 1 illustrates a vehicle 100 that has had a wrap 120 applied to the driver's side of the vehicle. Wraps 120 are becoming more and more popular as a way to advertise on vehicles. The intent is to have a flat, smooth film installation that appears like paint. Wrap 120 may include one or more thin film layers that are intended to be applied directly to the vehicle surface, such that the desired message appears painted on.

However, as illustrated, the doors of vehicle 100 have curvature 110, and the wrap 120 spans the gap 130 between the driver door and the back passenger door. While a position of the car can be automatically detected, such that wrap 120 can be automatically applied, and curvature 110 may be available from a CAD model of the vehicle 100, a system or method is needed to inspect the surface where wrap 120 will be applied-both pre and post application. Pre-application imaging may be helpful to detect and remove dust or debris before wrap 120 is applied. Post-application imaging may be helpful to quality check—e.g. identify and remove air bubbles and wrinkles.

As described herein, inspection may take place substantially immediately before or after a repair, for example using an imaging system. The image system may be mounted on a moving robot arm, on an unmanned aerial vehicle (UAV), or may have its own movement mechanism.

In some embodiments, a CAD model is not available, or an exact position of vehicle 100 is not available. It may then be suitable for an inspection system to, first, determine a topography 110 of vehicle 100, and provide that information for application of wrap 120. In some embodiments, multiple passes are conducted. For example, a first pass may identify potential areas of interest—e.g. areas of odd topography, a suspected defect, etc. A second pass may provide greater resolution of said topography or images of a defect. The additional information may be useful for providing information to a defect repair technician or system. In other embodiments, the second pass, or a third pass, is done after a repair to confirm that a defect has been repaired, and to understand how the repair has changed the surface e.g. if the surface is now acceptable.

Currently, much of the quality control process for aesthetic applications like vehicle wrapping is done by human technicians and is therefore subjective. Using a mobile vision system allows for a more objective quality control standard. In some embodiments, the system outputs location information of detected defects, defect classification (e.g. trapped debris or an air bubble) and/or defect severity. Based on combined information about a number of detected defects, the system may also provide an overall rating about the acceptability of a graphic film installation.

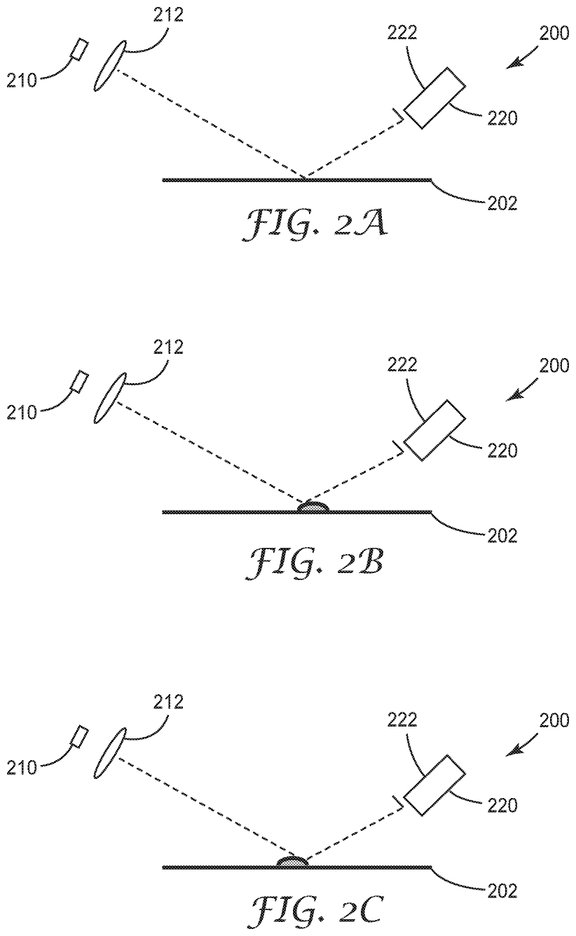

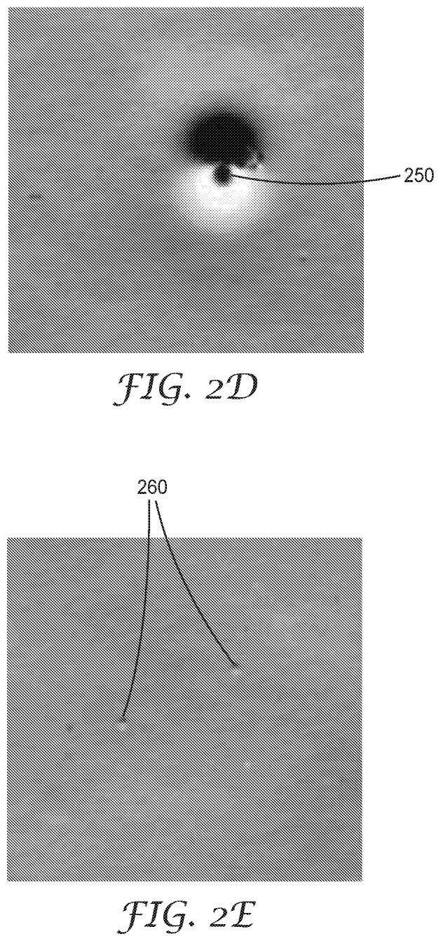

FIGS. 2A-2G illustrate operation of a line-scan array imaging system that may be suitable for embodiments herein, such as the inspection of vehicle 100 before or after application of wrap 120. FIG. 2A illustrates a linescan camera array system 200 with a linescan array 210, behind a lens 212. The array system is aimed at a surface 202 such that light from a light source 220 behind a knife edge 222, where everything is dark or gray. Array 210 captures a linear sequence of images that can be stitched together to form an image of a surface, as illustrated in FIGS. 2D and 2E. When linescan array 210 passes a defect or obstruction on the surface, light is deflected differently. If anything on the surface scatters or deflects the reflected light, then the image appears darker (if deflecting into the knife) or lighter (if deflecting away from the knife). The images in FIGS. 2D-2E demonstrate this effect for a large defect 250 and for more subtle defects 260.

FIG. 2D illustrates a defect on a surface, as detected using a linescan array. The light portion illustrated in FIG. 2D is caused as the system moves over the defect on the surface. A linescan array, such as that illustrated in FIGS. 2A-2C is very sensitive to light deflection. A robotics system may be useful for controlling a linescan array system because of the precise movement and control available using a robotic system.

In high reflective surfaces in particular, such as glossy paint surfaces, mirrors, etc., the angle of the light source and camera with respect to the sample are the key parameters for revealing surface attributes, similar to how humans observe surfaces at different angles to see different reflections of the surface. A linescan array, such as system 200, provides additional advantages, such as adjustable sensitivity by changing how close to the knife edge the imaging is aligned. However, it is expressly contemplated that systems and methods herein are useful for a variety of surfaces with different textures and reflectivity. The linescan array can be tuned to specific wavelengths to allow for maximum edge definition accuracy.

A linescan array system also works for both specular and matte surfaces. Imaging systems that can quantify surface parameters such as small changes in height indicating potential deviations from an expected topography can help fine tune an automated defect removal process. It is desired to sand only as much as possible to remove a defect, polish enough to achieve the needed surface finish, and manage device settings such as force applied, dwell time and movement speed to reduce haze and scratches. Systems and methods herein provide helpful feedback for improved robotic control.

FIGS. 2A-2E illustrate one configuration of line scan array imaging system that might be useful for imaging defects in a near-dark field mode of imaging. In some embodiments, a different configuration is used in a near-dark field mode of imaging.

In embodiments herein, it is envisioned that three passes may happen over a surface, first to obtain an initial topography of the surface, a second in a dark field mode, and a third in a near-dark field mode. However, it is possible that, in some embodiments as described herein, the first pass happens prior to a processing operation. Additionally, it is also contemplated that near-dark field imaging may happen prior to dark field imaging. Depending on an application, more or fewer passes, in the illustrated or different configurations, are also possible.

Once images are captured using imaging device 220, different analysis techniques can be applied to better characterize and quantify defect information. For example, deflectometry can be used to detect quantitative height value information, while the line scan image array on its own can only provide qualitative data of a defect height. However, it is noted that line scan image array data seems to be consistent with human vision perception. Deflectometry is particularly useful with highly reflective images, such that sufficient fringe patterns can be generated.

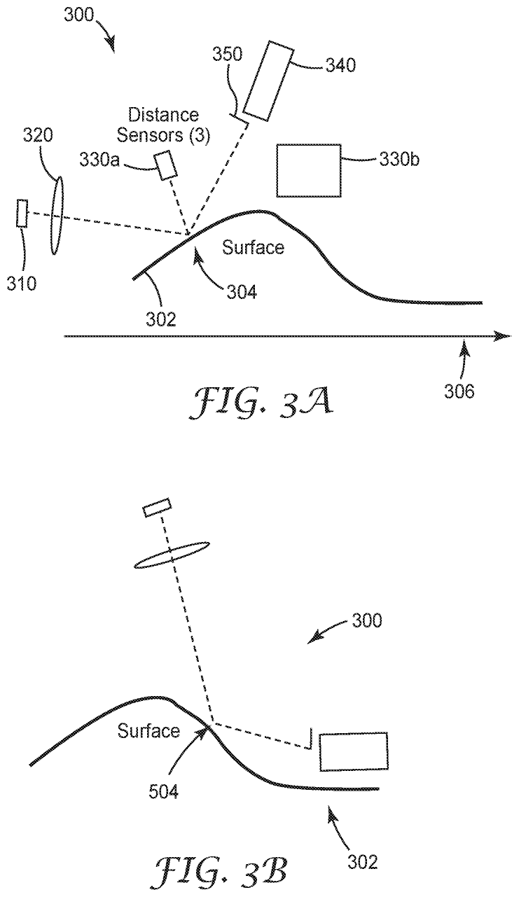

FIGS. 3A-3B illustrate a line-scan array imaging system for a curved surface. As illustrated in FIG. 1, many vehicles have curved surfaces. However, for a linescan array to take high fidelity images, and for post-image processing and quantification, it is necessary to have the sensing mechanism to be at a known position-both distance and angle, from the reflection point on the surface. While it may be possible to access a 3D model (e.g. a Computer-Aided Design or CAD model), such models may not be accurate enough, or may not be sufficient to know with sufficient precision where the reflection point is. It is desired to have a base understanding of surface topography, and then provide a linescan array with distance sensors to obtain a highly accurate topographical surface of the vehicle. However, it is also expressly contemplated that, in some embodiments, a system such as system 300 may be used to obtain the initial topography as well.

It is also necessary for the linescan array to be angled correctly with respect to the surface being imaged. It is desired that a right angle normal to the surface be present between the linescan array and the light source. In some embodiments herein, a distance sensor first passes over the worksurface, to obtain accurate distance and curvature information, followed by the linescan array in a second pass. In the second pass, the linescan array may be moved in order to achieve the desired position of a right angle normal to the surface at each point inspected. In other embodiments, the distance sensor is placed ahead of the linescan array. Based on feedback from the distance sensor, the linescan array position with respect to the worksurface is adjusted in-situ.

FIG. 3A illustrates a schematic view of an imaging system 300 imaging a surface 302. A linescan array 310, behind a lens 320, faces a surface 302, with the right angle between array 310 and light source 340 being orthogonal to surface 302 at point 304 as array 310 captures images of surface 302.

Imaging system 300 also includes a distance sensor, or distance sensor array. As many vehicles have surfaces with curvature in more than one direction, it is important to have distance information for at least the distance that the length of array 310 will pass through. As described above, in some embodiments a distance sensor travels separately from system 300, for example as illustrated by sensor position 330b. In some embodiments, sensor position 330b is representative of a real-time position of a sensor with respect to system 300 such that a sensor array moves, as indicated by arrow 306, across surface 302 ahead of system 300. Sensor position 330b illustrates an embodiment where a sensor array moves independently from system 300. However, it is expressly contemplated that a sensor array may be mechanically coupled In some embodiments, however, sensor position 330b is indicative of movement of the sensor array during a first pass, prior to system 300 traversing along path 306.

In some embodiments, a sensor array is mechanically coupled to system 300, as indicated by sensor position 330a, such that the sensor array travels along path 306 in a fixed position with respect to system 300. The entire system 300, with a sensor array in position 330a, may move across surface 302 in a first pass, so that distance sensors may capture accurate topography for surface 302, and then in a second pass so that system 300 may capture images of surface 302.

As illustrated in the transition from FIG. 3A to 3B, an orientation of system 300 changes in order to maintain a right angle at a normal to the point 304 being imaged. Based on information from a position sensor array, a robot arm, an UAV or other movement mechanism for system 300, rotates and moves system 300 to maintain a desired distance from, and orientation with respect to, surface 302. One sensor array is needed for a surface with zero Gaussian curvature, such as a cylindrical surface. However, multiple sensor arrays may be used in embodiments with non-zero Gaussian curvature surfaces, such as a spherical surface.

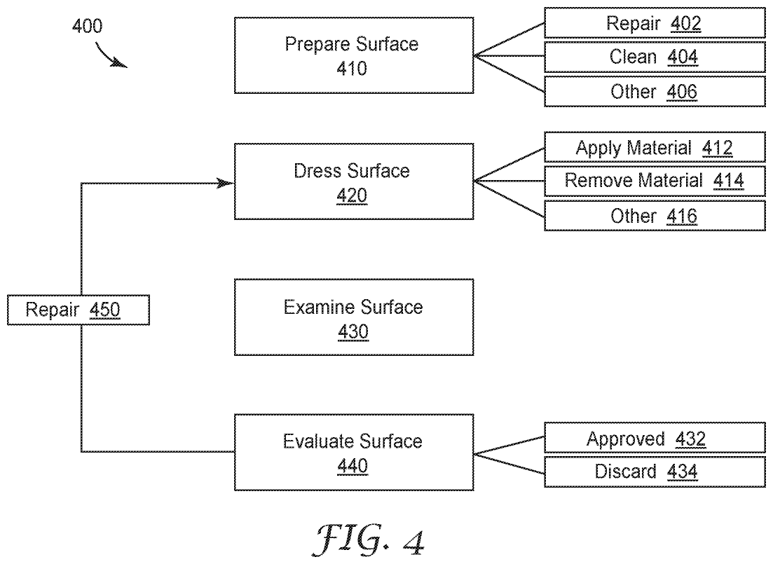

FIG. 4 illustrates a method of preparing and evaluating a surface after a material application in accordance with embodiments herein. Method 400 may be used for evaluating any suitable surface undergoing processing or that would benefit from imaging. A surface may be a flat surface, a curved surface, an irregular surface, or a surface with features such as corners, localized hills or valleys, etc.

In block 410, a surface is prepared for an operation. This may include getting a surface into a relative position with the imaging system, as indicated in block 402. This may involve moving the imaging system to the surface or getting the surface in a position known to the imaging system. In some embodiments the imaging system has a movement mechanism that allows for it to be moved in three dimensions to better approach a surface and follow a topography of the surface. In some embodiments, the imaging system is mounted on an end-of-arm of a motive robotic unit. In some embodiments, the imaging system is mounted on a remote controlled UAV. Preparing a surface may also include, as indicated in block 404, cleaning the surface or other preparatory procedures, as indicated in block 406.

In block 420, a surface is dressed. Dressing the surface may include applying a material, as indicated in block 412, removing a material, as indicated in block 414, or another operation, as indicated in block 416. It is also contemplated that, for some applications, the surface is not dressed, only imaged and examined.

In block 430, the surface is examined. The surface may be examined in real-time, for example as information is captured by an imaging system, in some embodiments. In other embodiments, a surface analyzer does not complete an analysis until the surface area of relevance is completely imaged. The captured images may be processed as described herein, or in another suitable manner to detect a defect or topography.

In block 440, the surface is evaluated. Based on an analysis of captured images, an evaluation of the suitability of the surface is done. The surface may be satisfactory and approved, as indicated in block 432 (e.g. for FIG. 1, air bubbles are sufficiently small or in locations where they are not easily seen); or discarded in block 434 (e.g. for FIG. 1, wrinkles are unacceptably large or the film has been stretched out of proportion), or can be repaired as in block 450 (e.g. air bubbles are detectable but repairable). The method may proceed back to block 420, as illustrated in FIG. 4, so the surface can be redressed, if applying more material 412, or removing material 414, addresses the issue.

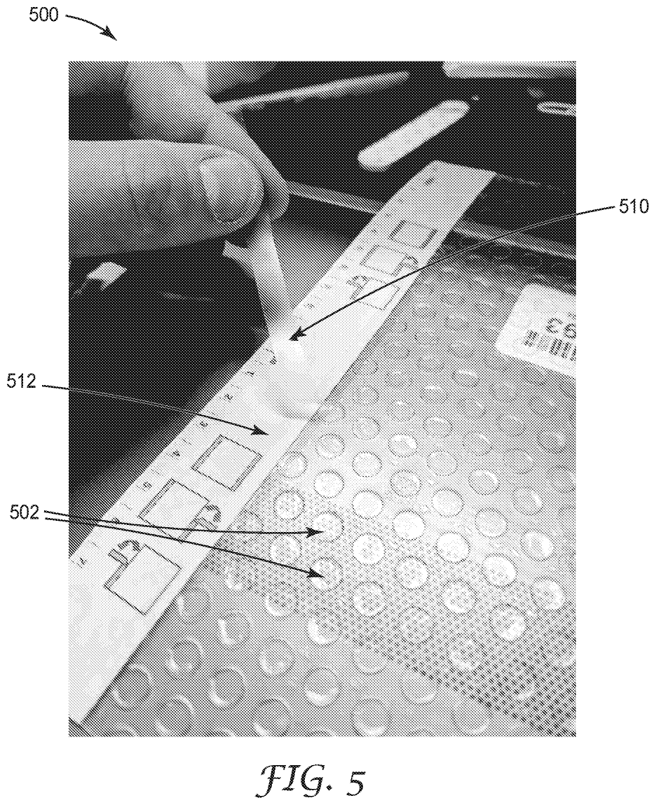

FIG. 5 illustrates a surface with a detected defect being addressed in one embodiment. Surface 500 is an Embossit™ dressing with an air bleed liner 510. The liner 510 is clear, as is the dressing 500. As illustrated, transparent liner 510 is present on only a portion of the dressing. Often just a strip remains over the interface between the handling bars and the polyurethane film. Currently, defective products are identified by hand. However, a system that can automatically pick out dressings with liners that have been insufficiently removed would be helpful. The manufacturing speed of such dressings is currently around 80 feet per minute. In some embodiments, a topography detection system may be stationary over a moving product line. The illustrated dressing is only about 1 millimeter high, with a width around a few millimeters. The liner is about 0.1 millimeters in height. The change in height from the presence of the liner may be enough to be detectable. Detection may cause an alert to sound such that the residual liner is removed in-situ, in some embodiments. In other embodiments, a location or a product number or another suitable identifier is logged and provided for a repair technician or an automated repair system.

Surface 500 includes a number of intended features 502. Liner 510 should have been removed from a paper handle 512. A topography detecting system, such as that described herein, may detect the presence of the liner either by detecting the change in height because of the presence of liner 510, or based on a difference in reflectance of liner 510 and the paper handle 510.

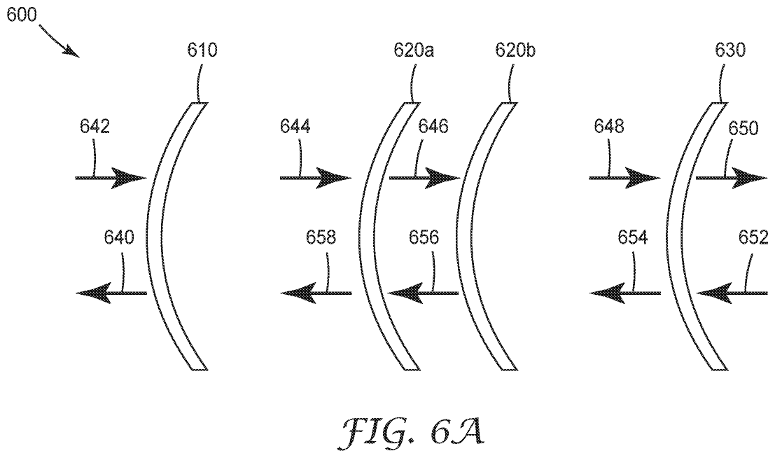

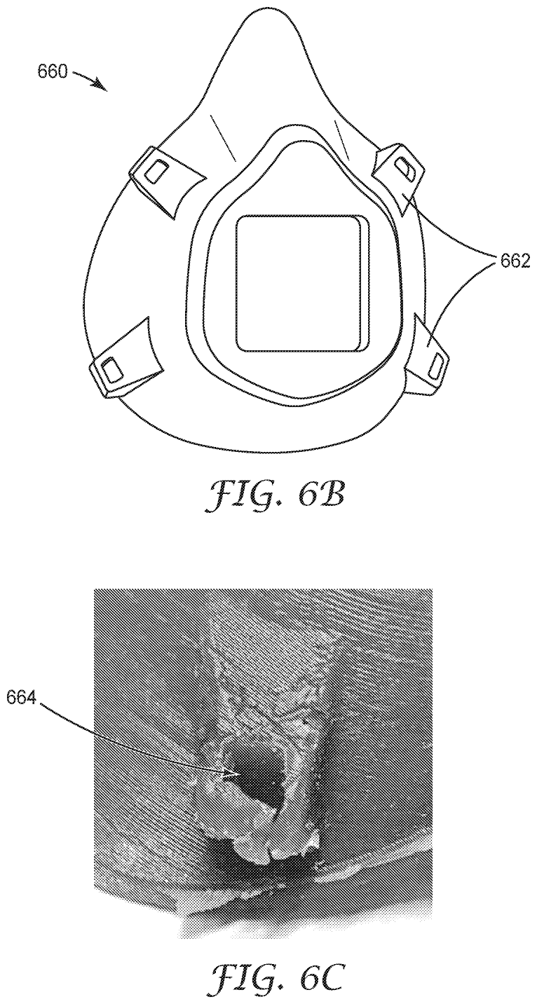

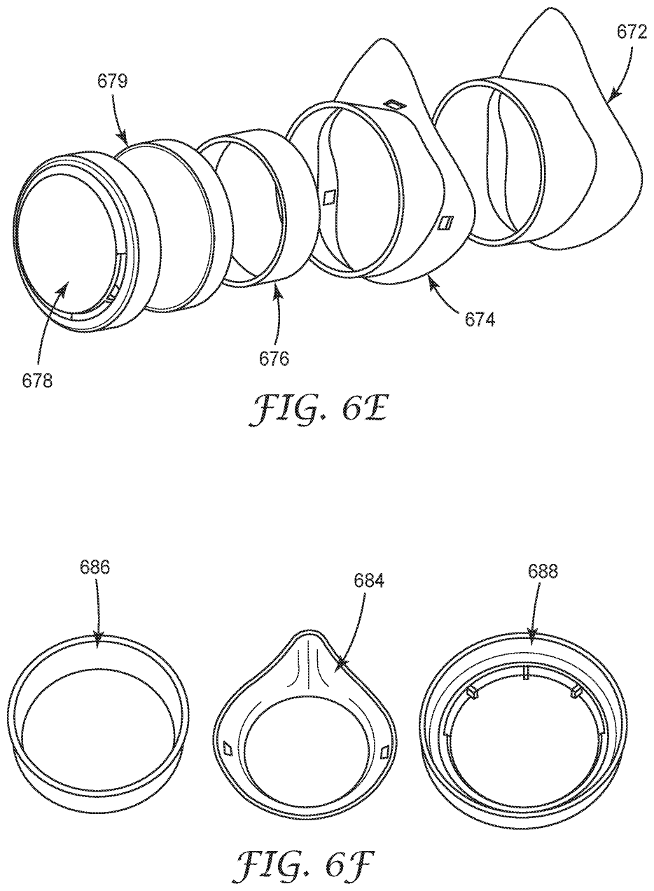

FIGS. 6A-6E illustrates assembly of a respirator that may be inspected using systems and methods herein. A respirator is designed to fit over the nose and mouth of a wearer and seal to the wearer's face, such that all air passes from the ambient environment, through layers designed to filter out different target contaminants, such that a user breaths in contaminant-free air. A respirator includes an assembly 600 of multiple layers. The layers may include, as illustrated in FIG. 6, a hydrophobic layer 610, one or more electrostatic layers 620a-b, and a biocompatible layer 630 that contacts the skin of a user. It is necessary that adjacent layers seal effectively such that air flows in accordance with the path 642-644-646-648 from ambient atmosphere 640 to an interior area 650, and back out along path 652-654-656-658. If a seal is incomplete, air will be forced out (or pulled in) through the incomplete seal. This results in a user being exposed to unfiltered air leaking through the gap in the layers.

FIG. 6B illustrates a CAD model that may serve as the basis for a 3D printed respirator mold. To form mold portion 660, a 3D printing system deposits polymer layers to form a mold shape to fit over the mouth and nose of a person to provide protection. Respirator portion 660 has multiple attachment points 662 that may receive fasteners. As illustrated in FIG. 6C, 3D printing can result in a mold with faulty attachment portions 664, for example as a result of poor molding or tearing.

FIG. 6C illustrates a CAD model of a number of components that, together, form a respirator. Portion 672 is molded, formed as described with respect to FIGS. 6A-6B or another suitable method. Portions 674, 676 and 678 are all 3D printed components. A filter 679 is placed over component 676 and held in place by component 678. If all 3D printed components are free of defects, air is forced through filter 679 so that contaminants are removed and the wearer is not exposed.

However, if there is a gap or pinholes in any of components 672-678, any defects that cause components 672-678 to incompletely seal together, or other defects such as faulty attachment points 662, the wearer of the respirator can inhale contaminated air. Systems and methods herein can be useful to detect such gaps along the curved surface of the 3D printed respirator, and the defect can be eliminated by design change or change to process parameters.

The need for a complete seal in between layers 610, 620, and 630 has been a cause for difficulty in 3D printing of respirators. Using a vision system like that described herein, a seal can be examined between layers as they are deposited, or of the finished respirator, such that gaps can be detected.

Similarly, other molded parts with quality control concerns may also benefit from imaging that is done during or after manufacture. It may be possible to detect weak or thin areas that may potentially fail.

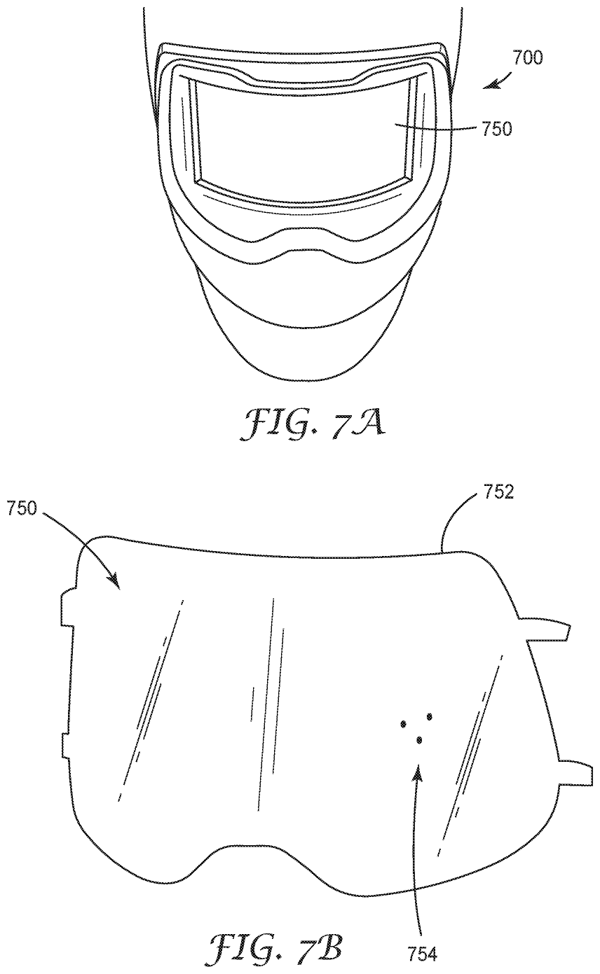

FIGS. 7A and 7B illustrates a glass welding helmet that may benefit from systems and methods herein. A welding helmet 700 has a glass shield 750. The shield 750 is a glass shield with curvature 752. The technique for forming curved glass shield 750 is sensitive to contaminants. Shield 750 has a protective film applied over the surface and the presence of debris on the glass surface 754 can create bubbles in the film. A topography imaging system may be able to detect debris before a film is applied, or may be able to locate air bubbles or imperfections. Air bubbles may be addressed by an installer, for example, applying pin holes to bleed out trapped air.

While air bubbles on a welding helmet shield is presented as one example, it is expressly contemplated that other defects may be detectable in molded or formed parts such as cracks, grazing, or minor imperfections. Quality of applied films may also be evaluated using topography detection systems described herein.

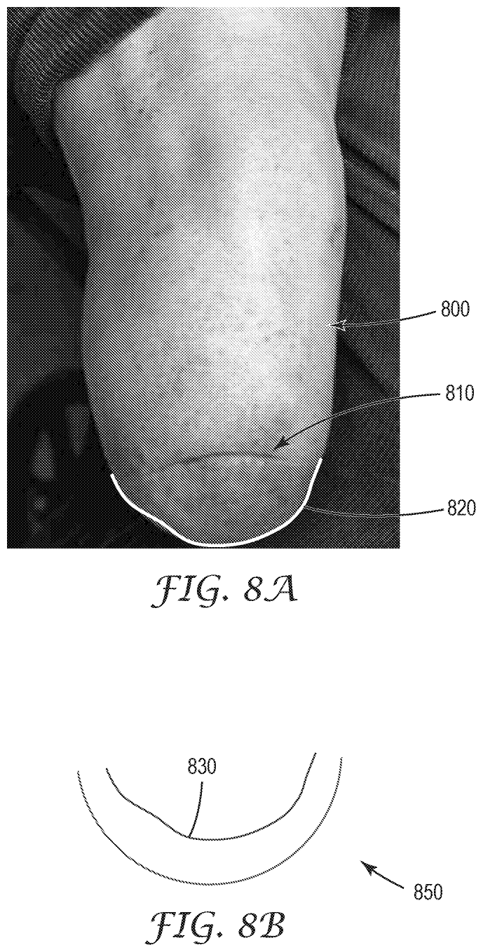

FIGS. 8A and 8B illustrate a prosthetic fitting that may benefit from systems and methods herein. Thus far, examples herein have described processes where material is added or removed to a surface and, thereafter, inspected. However, it is also expressly contemplated that vision systems such as those described herein may be useful for other applications regarding irregular surfaces.

Prosthetics are uncomfortable if the fit is not good. A wearer may experience chaffing, blistering, rashes or pain with a poor fitting prosthetic. Worse, poor fitting prosthetics can cost the health care system thousands of dollars per patient and can be life threatening if a new wound opens up.

The amputation site topography may also change over time as remodeling occurs, which may result in a previously good fitting prosthetic becoming uncomfortable over time. Currently, molds for prosthetics are made using plaster casting, which is time consuming. A solution is desired that allows for quick scanning of the limb stump, processing of the collected image data to obtain a 3D topography of the limb stump, and formation of a prosthetic that fits the detected topography. It may also be possible to better design a tighter fit that distributes weight to the appropriate areas. (e.g. remaining bone v. soft tissue).

FIG. 8A illustrates an amputee 800 being fitted for a prosthetic. The amputation process often leaves each end-of-limb with a unique topography 820, that may include features like fold 810, where skin was sewn back together. For this reason, prosthetics are often expensive and require customization to be comfortable. A mobile imaging system may capture topography 820 which can then be used as the basis for an interior topography 830 of a prosthetic 850. Using a topography imaging system to get an accurate 3D topography of limb 800 allows for features 810 to be fully captured and for a prosthetic interior 830 to be formed that fits the amputee comfortably. The prosthetic 850 may be 3D printed, for example based on topography 820, or may be molded or formed in another suitable method.

While prosthetic fitting is illustrated as one example in FIG. 8, it is expressly contemplated that other personalized medicine applications of a topography detection system are possible. For example, having a topography of a patient's body may assist in custom wound care treatment or a custom IV-securing mechanism.

Another example is scanning the topography of the soles of the feet to create custom orthotics or to select best fitting orthotics from available models to improve patient comfort. This is especially useful for patients experiencing diabetic foot ulcers.

Additionally, while systems are described herein as useful for the manufacture of personalized medical devices, it is also expressly contemplated that they may be used to quickly quality check, or verify that the correct device is going to the correct individual, but using an image system to scan the custom surface and compare the captured topography to an expected topography. If a match is not detected, the device has been mislabeled.

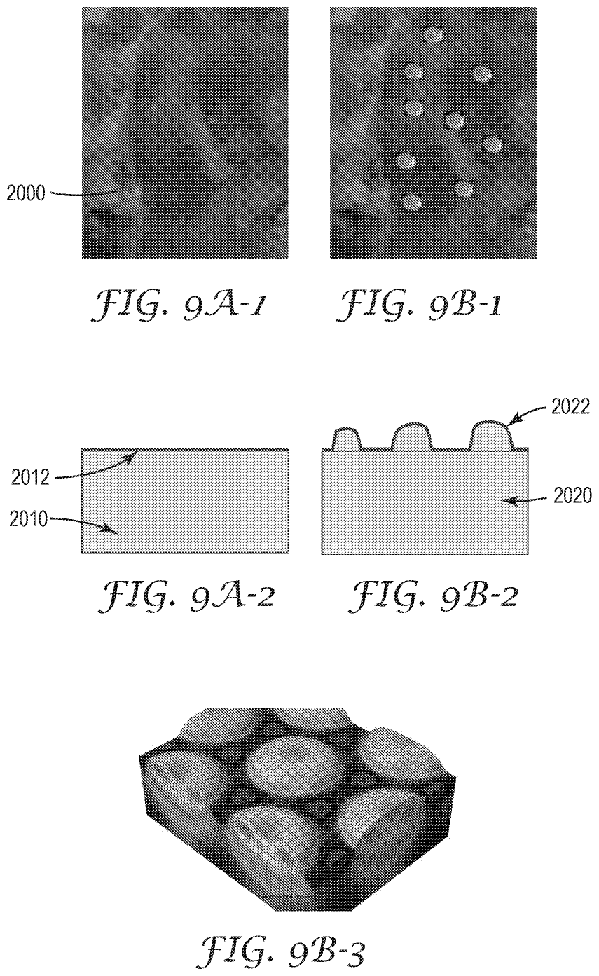

FIGS. 9A-9B illustrate tissue samples before and after a negative pressure treatment. Negative pressure wound therapy (NPWT) is a method of drawing out fluid and infection from a wound to help it heal. NPWT promotes healing by removing healing inhibitors, increasing blood flow, stimulating angiogenesis and granulation tissue and causing mechanical stress in the wound bed. FIGS. 9A-1 and 9A-2 illustrate an actual top-down view (9A-1) and schematic cutaway view (9A-2) of tissue before NPWT, where native tissue appears flat. In FIGS. 9B-1 and 9B-2, the native tissue has some “domes” that appear in the hours following NPWT. FIG. 9B-3 illustrates a finite element analysis model showing the contours of skin following NPWT.

Systems and methods may be used herein to measure the change in topography before and after NPWT to understand how the topography changed. Referring to FIGS. 9A-2 and 9B-2, tissue 2010 has a surface path length 2012 to traverse a surface of tissue 2010. This corresponds to a surface area of a patient's skin. After NPWT, the path length 2022 of post-treatment tissue 2020 has increased due to the swelling of tissue in different areas of tissue 2020. A strain measurement of the skin may be determined by comparing path lengths 2012 and 2022. For example, if path length 2012 is 2 cm, and path length 2022 is 2.5 cm, there is a 40% strain because of the swelling.

Currently, tissue strain is not easily measured without taking a biopsy. Computer modeling (such as that illustrated in FIG. 9B-3) is often used as a substitute. Systems and methods herein may provide a less invasive way to obtain a more accurate understanding of patient tissue strain. This may be used to iterate therapies for a patient and improve wound care.

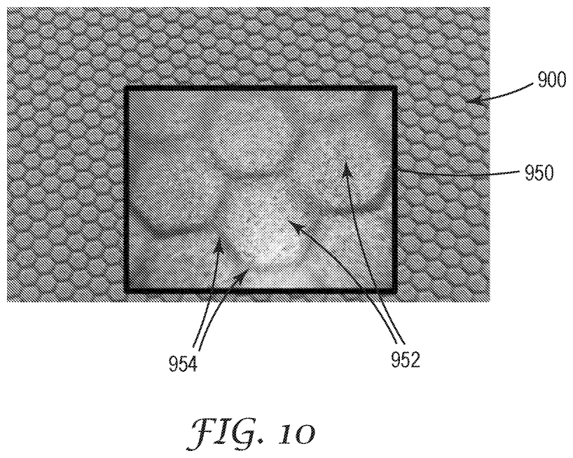

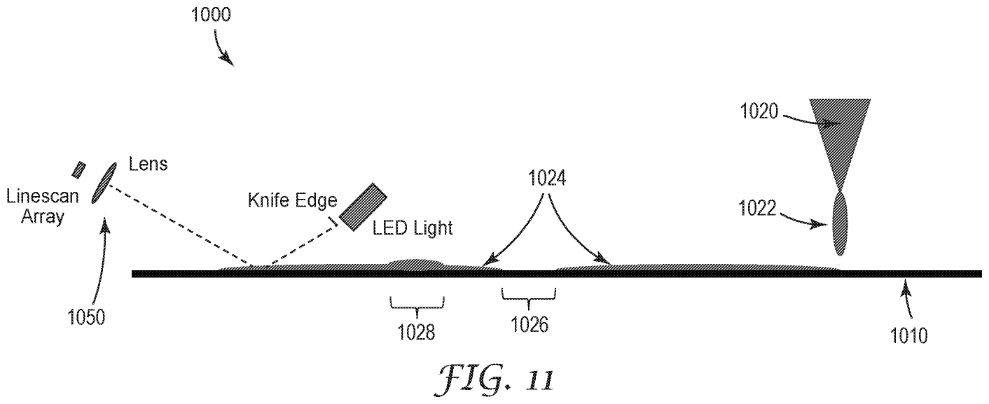

FIG. 10 illustrates a microreplicated surface that may be inspected using systems and methods herein. A surface 900 is formed of a number of structures 952 (as seen in the enlarged portion 950). Structures 952 may have channels 954 or spacing between them. In a microreplicated structure, there is an expected relationship between adjacent structures 952, with equivalent spacing 954, and a geometric alignment, as illustrated in image 900. In some applications, it is particularly important that the structures 952 and/or spacing 954 be precise and error free. FIG. 10 illustrates a TRIZACT™ abrasive surface that exhibits improved performance because of precise placement of structures 952 and channels 954.

Different microreplicated technologies may have different tolerances than others, but all may benefit from quality checking using imaging systems described herein, which can detect changes in height that are expected (e.g. channels 952) or unexpected (trapped debris, extra abrasive slurry, etc.).

Images of microreplicated surfaces may be processed by obtaining a binary pattern based on expected heights or density, with defects detectable as not fitting into expected height or density ranges.

Micro-replication (MR) applications rely on the existence of large fields of 3D features that are both hard to see but are assumed to be consistently present. Missing, damaged or mal-formed microscopic features could degrade the intended macro performance of the MR material.

Using topography detecting systems described herein, it may be possible to scan an undulating substrate onto which the MR is applied or formed, which should produce a distinctive imaged pattern that could be interpreted using Fourier Transform analysis and/or machine learning.

Patterns from a perfect product could be fingerprinted to provide known good samples, or such standards could be calculated using theoretical assumptions. Then, defects or drift/deviations from the ideal would perturb the imaged pattern.

Systems and methods herein contemplate a varying range of sensitivity, dependent on surface reflectivity as well as camera optics. For visually apparent features, it may be possible to detect changes in topography of 0.01 mm in size. Sensitivity can be increased as needed by changing camera settings relating to depth of field.

Metrics could be formed, based upon the degree of perturbation, that would indicate quality. Machine learning or Fourier analysis of the disruption to the pattern may then be used to characterize MR defects, anomalies, damage etc. anywhere along the material's lifecycle from manufacturing to end use.

Several different example surfaces have been described herein that could be inspected by systems and methods described herein. However, it is expressly contemplated that these are by example only, and that many other industries may benefit from similar surface inspection systems, such as idlers and structured rolls used in roll-to-roll processing, molded objects with or without thermo-formed layer, inspection of windshields and laminations thereof, embossing rolls, turbine blades with protective film, etc.

FIG. 11 illustrates an adhesive dispensing operation that may benefit from systems and methods herein. Adhesive dispensing is dependent on multiple variables, including temperature, speed of dispensing, speed of movement of a dispenser relative to a worksurface, proper mixing conditions, etc. A lot of effort goes into designing and troubleshooting dispensers 1020 so that adhesive 1022 is dispensed consistently onto a substrate 1010. However, as illustrated in dispensing operation 1000, it is possible to have an image system 1050 to follow dispenser 1020 to confirm that the right amount of adhesive is dispensed. While FIG. 10 illustrates the imaging system 1050 as traveling separately from dispenser 1020, it is also expressly contemplated that, in some embodiments, they travel together as one unit, with the light source and linescan array trailing the dispenser at a set distance.

It is possible that too much adhesive 1028 is dispensed in some areas and too little adhesive 1026 in others. For some applications, there is tolerance for some variability in adhesive dispensed. For other applications, gaps in adhesive can result in an insufficient seal and cause worksurface 1010 to be discarded. Therefore, a system 1050 that can inspect adhesive in real-time, between dispensing and curing, may allow for less waste as additional adhesive can be added, excess adhesive can be smoothed out, or other measure may be taken to address issues before the adhesive cures.

Similarly, while a flat surface 1010 is illustrated for convenience, it is also contemplated that more or less adhesive may be needed for a curved surface. While it is illustrated in FIG. 10 that an imaging system 1050 travels behind an adhesive dispenser, it may also be possible, in some embodiments, for the imaging system 1050 (or a second system) may travel ahead of dispenser 1020. A forward imaging system may provide accurate upcoming topography information so that a dispenser can adjust metering speed accordingly.

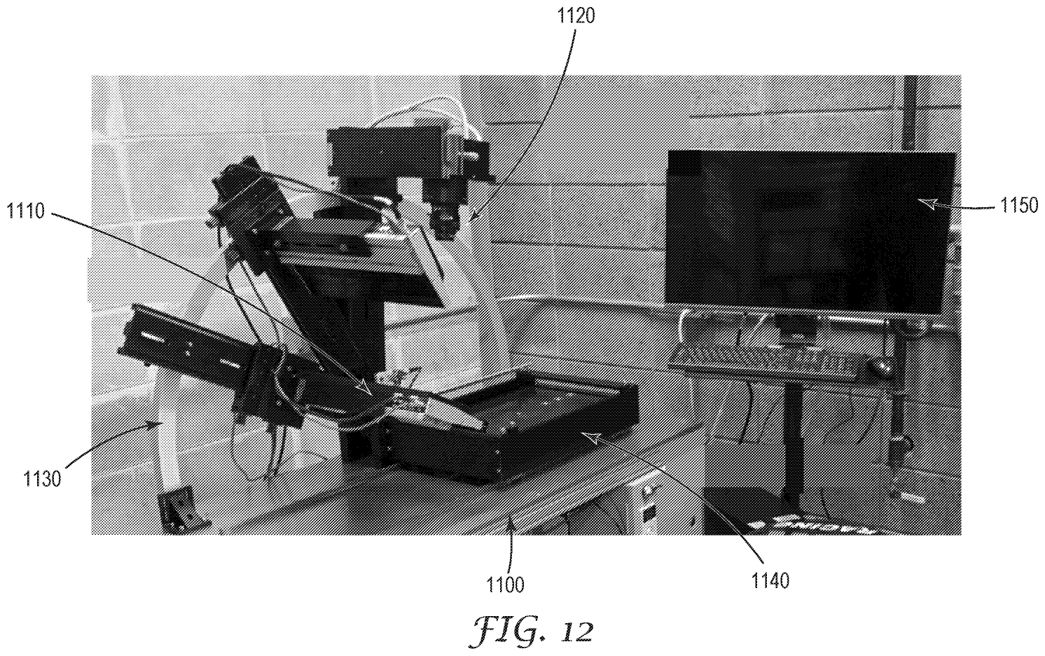

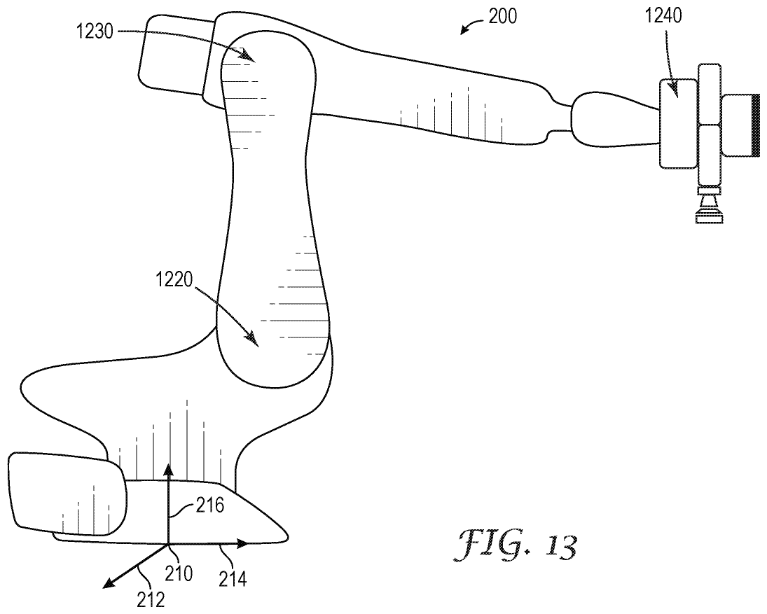

FIG. 12 illustrates an imaging system in accordance with embodiments herein. Imaging system 1100 is controlled by a controller 1150, which can receive instructions from an operator, for example using the illustrated keyboard. However, in some embodiments, system 1100 is automatically controlled by controller 1150, for example based on information received from a distance/position sensor or another source.

A linescan array 1120 images a surface 1140 which, in some embodiments, moves with respect to system 1100. However, it is expressly contemplated that, in some embodiments, a worksurface remains stationary and system 1100 is mobile. Light sources 1110 is directed toward surface 1140, so that light is reflected toward linescan array 1120.

An orientation component 1130, illustrated as a curved rail, may be used to maintain a desired orientation between light sources 1110 and linescan array 1120, while changing an orientation of system 1100 with respect to a worksurface 1140. This may be helpful in embodiments where surface 1140 has curvature, to maintain a desired orientation of normal to a right angle formed by one of lights 1110 and linescan array 1120. In the illustrated embodiment, orientation component 1130 operates independently to change the angle of light sources 1110 and imaging device 1120 with respect to surface 1140. This may be preferred as the optimum arrangement to reveal and characterize a defect may differ based on the optical properties of the surface as well as the light incident angle and camera position.

FIG. 13 illustrates a motive robot unit on which a topography mapping unit can be mounted in accordance with embodiments herein. A motive robot arm 1200 has several different pivot points 1220, 1230, 1240 that allow for freedom of movement. Depending on the design (robot 1200 is shown for illustrative purposes, not as a limitation), each of these may allow for movement in 1, 2 or three degrees of freedom. Additionally, robot arm 1200 may be stationary, at a point 1210, or point 1210 is indicative of an attachment to a movement mechanism—e.g. wheels or a rail system that allows for movement in 1, 2 or 3 dimensions. A topography detection system 1250 is mounted at an end-of-arm position in the illustrative embodiment. However, it is expressly contemplated that system 1250 may be positioned elsewhere, in some embodiments, e.g. behind or in front of a dispensing system.

Robot unit 1200 allows for a topography mapping system to travel across a worksurface. As noted above, in some embodiments, a linescan array may have a narrow range of high fidelity mapping. Robot unit 1200 may therefore guide system 1250 across a surface in a grid pattern until an entire area of interest has been mapped adequately.

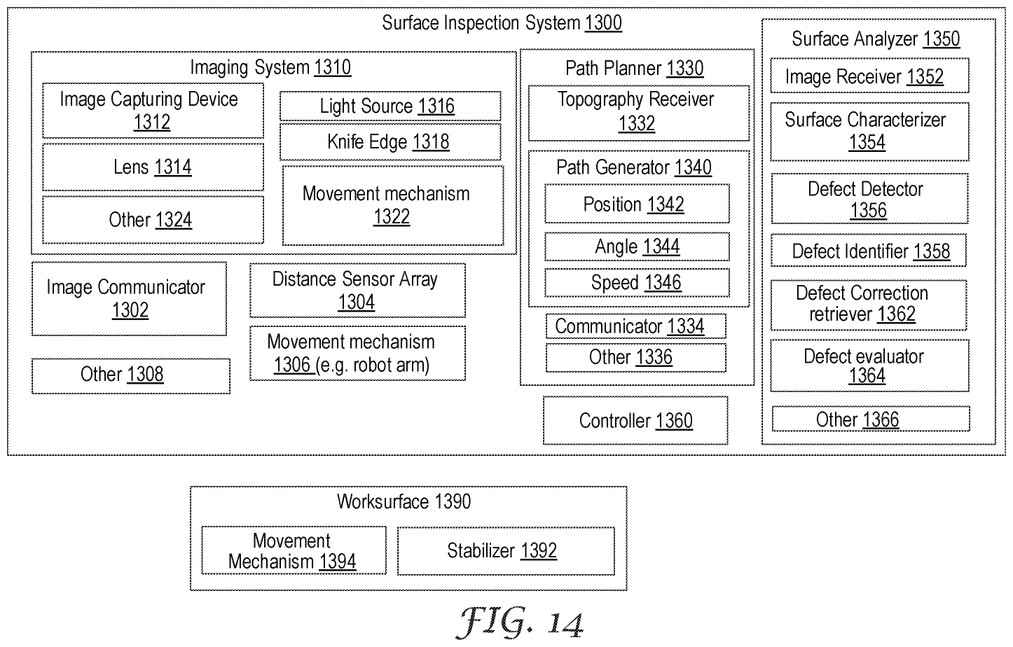

FIG. 14 illustrates a surface imaging system in accordance with embodiments herein. A surface imaging system 1300 may be used to capture images of a worksurface 1390. Worksurface 1390 may be a flat surface, a curved surface, an irregular surface, a surface having features like corners, indentations, hills and/or valleys. Worksurface 1390 may have curvature in one or more directions. Surface inspection system 1300 may be useful for imaging surface 1390 pre or post surface processing. Surface inspection system 1300 illustrates a number of components that may be useful for inspecting a surface 1390 using imaging system 1310. However, it is expressly contemplated that other components 1308 may also be useful, in some embodiments.

Surface inspection system 1300 includes an imaging system 1310 that captures images of worksurface 1390. Images are captured by a linescan array 1312. A lens 1314 may be used to focus the cameras in the linescan array 1312. Linescan array 1312 is aimed at worksurface 1390 such that light, from a light source 1316 reflects off worksurface 1390 to linescan array 1312. A knife edge 1318 is placed in front of light source 1316. Imaging system 1310 may include other features as well, such as a second lens 1314, or a second light source 1316. Imaging system 1310 includes a movement mechanism, in some embodiments, such that imaging system 1310 can move with respect to a worksurface 1390 so that a normal is maintained with respect to the right angle formed by linescan array 1312, worksurface 1390, and light source 1316. Movement mechanism 1322 may rotate imaging system 1310, raise or lower imaging system 1310 with respect to worksurface 1390, or otherwise adjust a relative position of imaging system 1310 with respect to worksurface 1390. Movement mechanism 1322 may be part of, or coupled to, a robotic arm, in some embodiments. Imaging system 1310 may capture images of worksurface 1390, which may then be stored or processed, for example by surface analyzer 1350. Imaging system may also include other components 1324.

In some embodiments, an image captured by imaging system 1310 is communicated to another device, using image communicator 1302. For example, a captured image may be sent to a storage component, or provided on a display for review.

In some embodiments, surface inspection system 1300 includes a distance sensor 1304. Sensor 1304 may be a distance sensor array, in some embodiments. Distance sensor array 1304 may be coupled to imaging system 1310, such that it moves with imaging system 1310, in some embodiments. Imaging system may move ahead of imaging system 1310, with imaging system 1310 or behind imaging system 1310. In other embodiments, distance sensor array 1104 moves independently of imaging system 1310. Distance sensor array 1304 passes over worksurface 1390, for example using movement mechanism 1306, which may be coupled to, or separate from, movement mechanism 1322. Distance sensor array 1304 captures detailed topography information for worksurface 1390 so that imaging system 1310 can pass over worksurface 1390 and take highly accurate images, from the desired orientation.

Distance information, captured from distance sensor array 1304, is provided to path planner 1330, which calculates a path for imaging system 1310 to travel over worksurface 1390. Topography receiver 1332 receives distance information and provides topography information to path planner 1330. Based on the worksurface topography, path generator 1340 generates a path for imaging system 1310 to travel. A path includes a position 1342 of imaging system 1310 relative to worksurface 1390, ad an angle 1344 that imaging system 1310 needs to rotate in order to maintain a position normal to worksurface 1390. Position 1342 refers to a spatial position required to keep a desired distance between imaging system 1310 and worksurface 1390.

In some embodiments, movement mechanism 1306 is a robot arm, such that imaging system 1310 is attached to a robot end effector. In some embodiments, 3 or more distance sensors are included in a sensor array 1304. Preferred sensors could be, for example, LM Series Precision Measurement Sensor from Banner Engineering, Keyence CL-3000 Series Confocal Displacement Sensor from Keyence.

The sensors of sensor array 1304 may be spaced across the cameras effective field of view, to provide a sparse 3D distance map.

Many surfaces are curved in at least two directions. Therefore, using a standard line array, it may be that only a center section of the image will be valid. Suitable image processing can be done to identify valid regions, for example, using the image itself or using information from the 3D surface mapping. However, it is expressly contemplated that, in some embodiments, a 3D camera scanning system is used to fully map the surface. Such systems are available from companies such as Cognex, Keyence, and LMI.

From the 3D map, a path can be planned for a robot unit with the imaging system. The path is calculated so that at each point, the imaging system is normal to the surface. A robotic arm can precisely control angle and distance to ensure high quality imaging.

In some embodiments, path planner 1330 is configured to allow for a single pass of imaging system 1310 and distance sensor array 1304 over worksurface 1390. For example, topography receiver 1332 can receive feedback from distance sensor array 1304 substantially in real-time, and path generator 1340 generates a path and provides instructions to movement mechanism 1322 to change a position 1342, angle 1344 or speed 1346 of imaging system 1310 along a path. The distance sensor feedback is provided, path generated and communicated back to movement mechanism 1322, using communicator 1334, and imaging system 1310 is moved accordingly in the time it takes for imaging system to traverse a distance between imaging system 1310 and distance sensor array 1304. For example, if distance sensor array 1304 is coupled to imaging system 1310 with a separation of 3 inches in between, then the information is transmitted, path returned, and imaging system adjusted in the time it takes for imaging system 1310 to travel 3 inches.

In other embodiments, a two-pass system is used, such that, in a first pass, distance sensor array 1304 retrieves topography information, which is provided to path planner 1330, which generates and communicates, using communicator 1334, the path back to movement mechanism 1322, which implements the positions 1342 and angles 1344 for imaging system 1310 during the second pass.

Path planner 1330 may also have other features 1336.

Images captured by imaging system 1310 are provided to surface analyzer 1350 which, in some embodiments, provides analysis regarding surface parameters of worksurface 1390, such as whether the surface has an unexpected deviation in height—for example caused by too much or too little surface processing. Surface analyzer 1350 may also provide relative height information such that a topography of a surface can be mapped. Images are received by image receiver 1352. Image information may be received in substantially real-time from linescan array 1312, in some embodiments, and image receiver 1352 may assemble an image from the array signals received. Once the images of worksurface 1390 are collected, they can be viewed by a human operator, or automatically analyzed for quality control concerns.

A defect detector 1356 may, based on images from image receiver 1352, identify defects on worksurface 1390. Defects may be too much material present (e.g. trapped debris, too much adhesive dispensed, etc.) or too little material present (e.g. a scratch, a crack, too little adhesive dispensed, etc.).

A defect identifier 1358 may, based on information from defect detector 1356 and surface analyzer 1350, identify a detected defect as a particular type of defect—e.g. as a scratch versus a crack.

A defect evaluator 1364 evaluates the identified defect for severity and whether it can be mitigated, such as by bleeding air from a detected air bubble, or repaired, such as by adding additional adhesive to fill a gap, for example.

A surface characterizer 1354 may, based on the results of analyzer 1350 and defect evaluator, output an indication of a quality of worksurface 1390 post processing. For example, the surface characterizer 1354 may determine that a surface 1390 is unacceptable, but repairable.

A defect correction retriever 1362 may, based on an indication that a defect can be mitigated, retrieve a defect mitigation procedure. For example, a location of a detected air bubble may be provided to a bleed system, for example, which may puncture the bubble based on the detected location. Or a location of excess dispensed adhesive may be provided to a wiping unit which may smooth or remove the excess.

Surface analyzer may have other features or functionality 1366.

Information from analyzer 1350 may be provided to controller 1360, which may adjust one or more parameters for the next operation to reduce future defects. For example, multiple gaps in dispensed adhesives indicate that a viscosity may be higher than anticipated and a metering speed should be increased to compensate.

Controller 1360 may also provide control signals to components of surface inspection system 1300, for example for movement mechanism 1322 to adjust a position or angle of imaging system 1310, for imaging system 1310 to begin capturing an image, or for distance sensors to begin capturing topography information.

Systems and methods have been described herein for scenarios where a worksurface is stationary during topography or imaging collection. However, it is expressly contemplated that systems and methods herein may also be applicable to embodiments where worksurface 1390 is moving. Imaging system 1310 may also be moving, either in the same or different direction of worksurface 1390, or imaging system 1310 may be stationary. In such embodiments where worksurface 1390 is mobile, it may have a movement mechanism 1394, such as a conveyor belt or wheels, and may also have one or more stabilizers 1392 to keep worksurface 1390 stable during imaging.

In some embodiments, a custom imaging lens includes telecentric imaging with a compact design is used to improve operation.

In some embodiments, the light source is a diffuse LED light with a knife edge. In another embodiment, the light source includes a small LCD display with individually addressable pixels, which may allow for sensitivity to be changed with no mechanical adjustments. In some embodiments, the knife edge has an automated height adjustment mechanism.

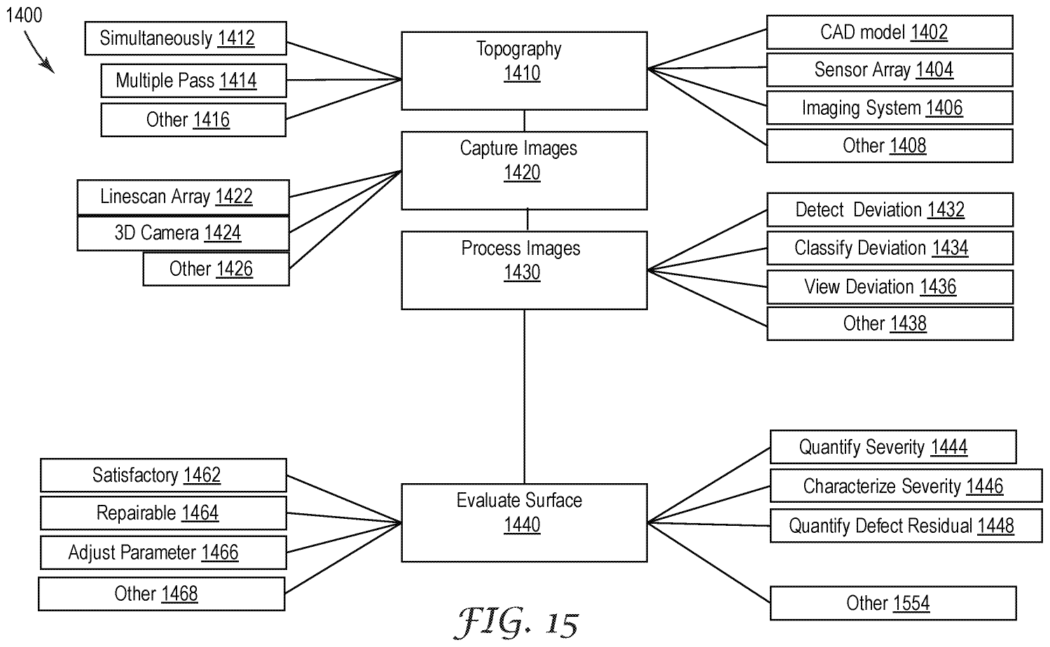

FIG. 15 illustrates a method of evaluating a worksurface in accordance with embodiments herein. Method 1400 may be used with any systems described herein, or another suitable system that images and analyzes images of a worksurface. The worksurface in question may be a flat surface, a curved surface, or a surface containing features such as hills, valleys, corners, indentations, etc.

In block 1410, a topography of a worksurface is obtained. In some embodiments, this includes retrieving a 3D model, such as a CAD model 1402. However, a CAD model 1402 is often not completely accurate with respect to surface topography, as paint coatings can be uneven, trapped debris can cause bumps, etc. and a vehicle may not be perfectly oriented or positioned in space. Therefore, in order to get high quality images of the surface, it is necessary, in some embodiments, to use a sensor array 1404 to get an accurate topography of the surface, particularly as many surfaces have curvature in multiple directions, corners, indentations, raised features, etc. For example, many vehicles have surfaces that curve in at least two directions, aka “complex curves,” or limb stumps may have asymmetrical curvature due to healing, adhesive may not be dispensed completely smoothly, etc. In some embodiments, it is necessary to first obtain a topography before imaging can take place, such that method 1400 proceeds in a multiple pass process 1414. However, it may be possible to obtain an accurate topography at the same time imaging is happening, either simultaneously 1412, or with an imaging system following a sensor array 1404. Other embodiments 1416 are also possible.

In another embodiment, it may be possible to obtain an accurate topography using an imaging system 1406, for example using an imaging system that also detects topography on the surface. Other suitable systems 1408 may be used to obtain a surface topography.

In block 1420, images are captured. Images can be captured using a linescan array 1422 at a known distance from the surface, in some embodiments. In some embodiments a 3D camera 1424 is used. For curved surfaces, it is necessary for an imaging device to be at a known distance from the surface at all times during a scan. Therefore, in some embodiments capturing images, in block 1420, includes an imaging device traveling along a path such that a set distance and/or orientation is maintained with respect to the surface being imaged. Other suitable imaging devices may be used, as indicated in block 1426.

In block 1430, captured images are processed to obtain information about the surface. A deviation from an expected topography may be detected, as indicated in block 1432, for example by detecting that a change in height is greater or less than expected. A detected deviation may be classified, as indicated in block 1434, for example as too much material being present, or too little. An image of the detected deviation may be presented for inspection, as indicated in block 1436, by a human operator. Other processing may be done to generate other useful views or address detected defects, as indicated in block 1438.

In block 1440, the suitability of a surface is evaluated. Evaluating the surface may be done manually, for example providing images to a human operator who indicates whether the surface is satisfactory 1462, whether it can be repaired, as indicated in block 1464 and/or whether a parameter needs to be adjusted for future operations, as indicated in block 1466, e.g. lower or higher force for a material removal operation, faster or slower dispensing speed, etc. Other suitable actions may be taken, as indicated in block 1468.

Alternatively, or additionally, the repair may be evaluated quantitatively by an image analyzer. Identified defects may be evaluated for severity, as indicated in block 1444. Severity may be evaluated on a defect-by-defect basis, or based on a holistic view of the surface—e.g. one large defect may be as problematic as ten small ones. Defects may also be characterized automatically by an image analyzer, as indicated in block 1446, to determine a severity of each individually, or in the context of the surface holistically. In embodiments where material is removed—e.g. air released from an air bubble, the residual defect may be examined, as indicated in block 1448. Other characteristics may also be quantified, as indicated in block 1454.

In some embodiments, at least some of the steps in method 1400 are completed automatically. For example, in a two-pass system, a second imaging pass may begin automatically after topography is obtained and a path planned for the imaging system. Additionally, processing of images may be done as soon as they are received, or even in-situ as imaging data is received, from a linescan array system. The worksurface may also be evaluated once images are processed. Instructions for components to conduct each of the steps or analyses illustrated in FIG. 11 may be provided by a robot controller. The instructions may include movement instructions for different components, including direction, speed, orientation, etc.

Method 1400 may need to be executed multiple times during a surfacing operation. For example a typical defect repair process for a vehicle includes (1) defect location and pre-inspection, (2) sanding, (3) wiping, (4) polishing, (5) wiping and (6) final inspection. Imaging may be needed in steps (1) and (6) and, based on imaging in (1), a sanding recipe may be selected to address a particular defect. Similarly, in the air bubble example of FIG. 1, instead of steps 2-5y, the intermediate steps may be puncturing the air bubble and smoothing the area.

After intermediate surfacing operations, a surface area may again be imaged so that a defect residual may be detected, characterized and quantified to determine whether additional repair is needed.

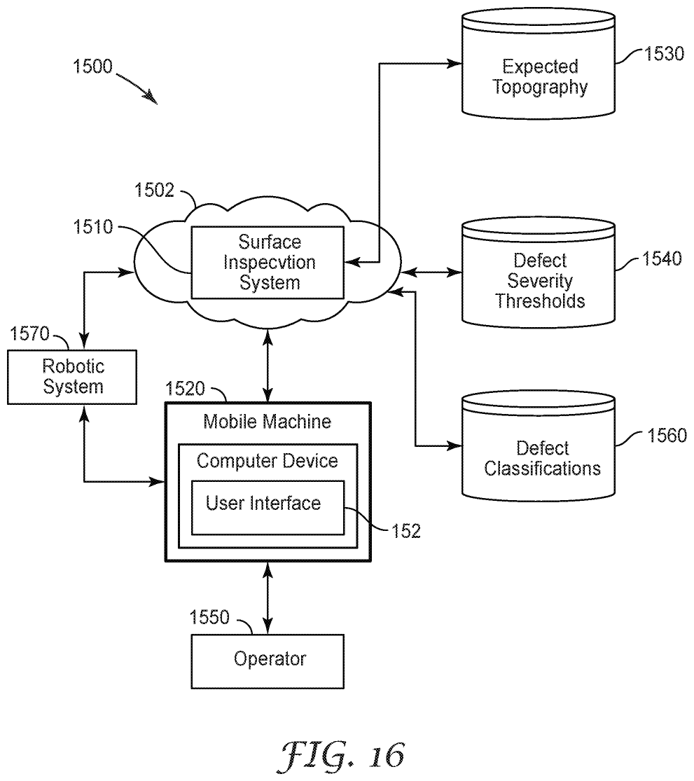

FIG. 16 is a surface inspection system architecture. The surface processing system architecture 1500 illustrates one embodiment of an implementation of a surface inspection system 1510. As an example, surface inspection system 1500 can provide computation, software, data access, and storage services that do not require end-user knowledge of the physical location or configuration of the system that delivers the services. In various embodiments, remote servers can deliver the services over a wide area network, such as the internet, using appropriate protocols. For instance, remote servers can deliver applications over a wide area network and they can be accessed through a web browser or any other computing component. Software or components shown or described in FIGS. 1-15 as well as the corresponding data, can be stored on servers at a remote location. The computing resources in a remote server environment can be consolidated at a remote data center location or they can be dispersed. Remote server infrastructures can deliver services through shared data centers, even though they appear as a single point of access for the user. Thus, the components and functions described herein can be provided from a remote server at a remote location using a remote server architecture. Alternatively, they can be provided by a conventional server, installed on client devices directly, or in other ways.

In the example shown in FIG. 16, some items are similar to those shown in earlier figures. FIG. 16 specifically shows that a surface inspection system 1510 can be located at a remote server location 1502. Therefore, computing device 1520 accesses those systems through remote server location 1502. Operator 1550 can use computing device 1520 to access user interfaces 1522 as well.

FIG. 16 shows that it is also contemplated that some elements of systems described herein are disposed at remote server location 1502 while others are not. By way of example, storage 1530, 1540 or 1560 or robotic systems 1570 can be disposed at a location separate from location 1502 and accessed through the remote server at location 1502. Regardless of where they are located, they can be accessed directly by computing device 1520, using system 510, through a network (either a wide area network or a local area network), hosted at a remote site by a service, provided as a service, or accessed by a connection service that resides in a remote location. Also, the data can be stored in substantially any location and intermittently accessed by, or forwarded to, interested parties. For instance, physical carriers can be used instead of, or in addition to, electromagnetic wave carriers.

It will also be noted that the elements of systems described herein, or portions of them, can be disposed on a wide variety of different devices. Some of those devices include servers, desktop computers, laptop computers, imbedded computer, industrial controllers, tablet computers, or other mobile devices, such as palm top computers, cell phones, smart phones, multimedia players, personal digital assistants, etc.

It is also noted that systems and methods herein may serve to collect data to teach a controller to better detect, classify and respond to a detected topography. A non-exhaustive list of machine learning techniques that may be used on data obtained from systems herein include: support vector machines (SVM), logistic regression, Gaussian processes, decision trees, random forests, bagging, neural networks, Deep Neural Networks (DNN), linear discriminants, Bayesian models, k-Nearest Neighbors (k-NN), and the gradient boosting algorithm (GBA). During operation, the trained behavioral model can then better characterize a topography determine an appropriate next step based on the characterization.

As described herein, in different embodiments, a system has tunable sensitivity based on camera specifications, aperture settings, lens stack specifications and lighting. A depth of field is adjusted based on lens effective diameter and focal length. depth of field refers to object space and depth of focus to image space. The field of view is that part of the object that is being examined, and the focus is the point at which parallel rays converge after passing through a lens.

It has been described herein that a system maintains a distance from a surface during imaging/topography mapping. In some embodiments, there is some tolerance for a change in distance, for example due to machine instability, jostling, imprecision, movement speed, etc. The amount of movement that may be tolerated may vary, for example based on the sensitivity desired in the resulting topography measurement.

Additionally, while systems and methods have been described herein that measure a topography to detect surface irregularity. However, it is also contemplated that systems and methods herein may also detect and measure surface features, such as glossiness, glass clarity, surface haze, etc. that change a physical appearance of a surface without changing the topography.

Systems and methods described herein expressly contemplate a linescan imaging system. However, it is expressly contemplated that other imaging devices may be suitable, such as an area-scan array, a 3D imaging camera, etc.

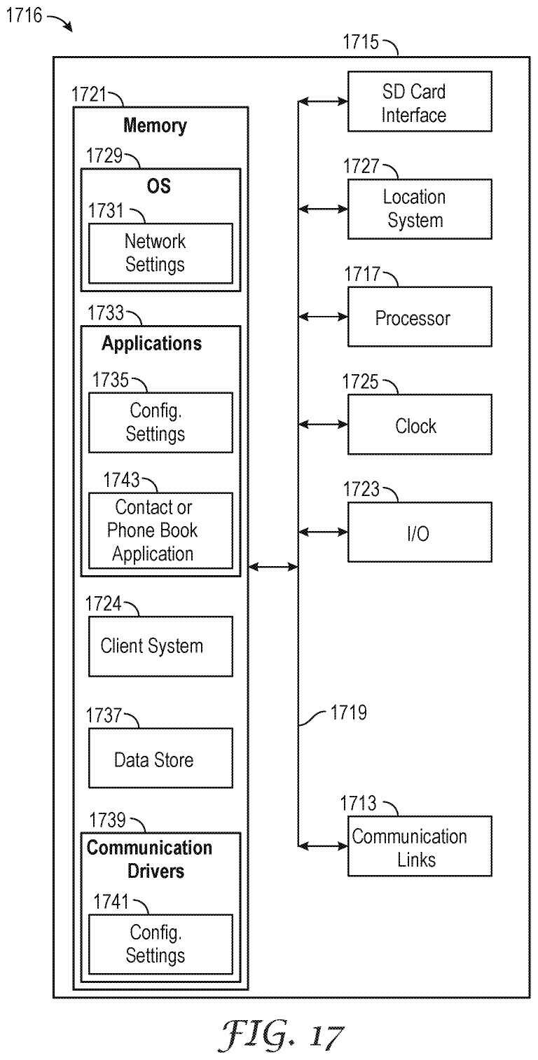

FIGS. 17-19 show examples of computing devices that can be used in embodiments shown in previous Figures.

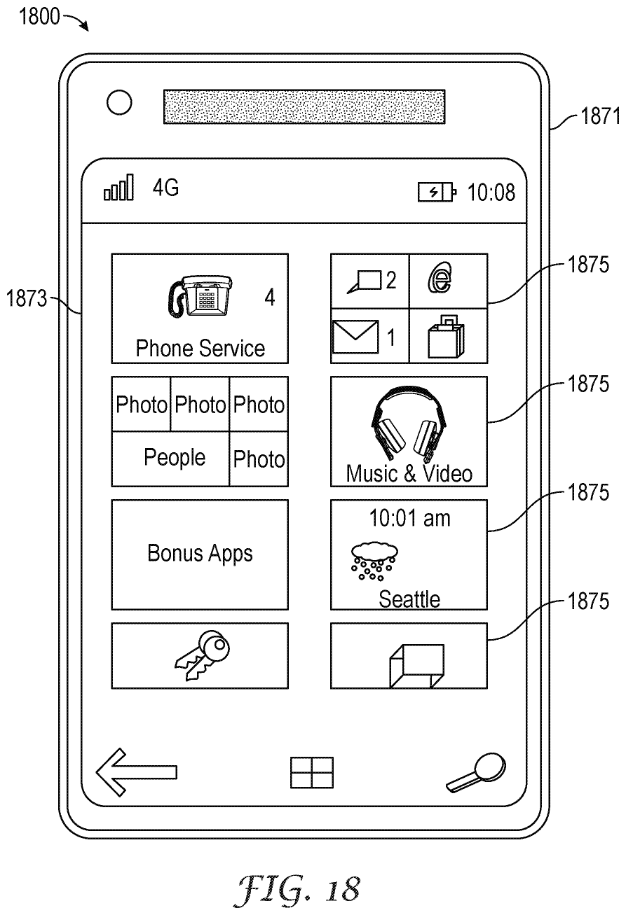

FIG. 17 is a simplified block diagram of one illustrative example of a handheld or mobile computing device that can be used as a user's or client's handheld device 1616 (e.g., as computing device 1520 in FIG. 16), in which the present system (or parts of it) can be deployed. For instance, a mobile device can be deployed in the operator compartment of computing device 1520 for use in generating, processing, or displaying the data. FIG. 18 is another example of a handheld or mobile device.

FIG. 17 provides a general block diagram of the components of a client device 1616 that can run some components shown and described herein. Client device 1616 interacts with them, or runs some and interacts with some. In the device 1616, a communications link 1613 is provided that allows the handheld device to communicate with other computing devices and under some embodiments provides a channel for receiving information automatically, such as by scanning. Examples of communications link 1613 include allowing communication though one or more communication protocols, such as wireless services used to provide cellular access to a network, as well as protocols that provide local wireless connections to networks.

In other examples, applications can be received on a removable Secure Digital (SD) card that is connected to an interface 1615. Interface 1615 and communication links 1613 communicate with a processor 1617 (which can also embody a processor) along a bus 1619 that is also connected to memory 1621 and input/output (I/O) components 1623, as well as clock 1625 and location system 1627.

I/O components 1623, in one embodiment, are provided to facilitate input and output operations and the device 1616 can include input components such as buttons, touch sensors, optical sensors, microphones, touch screens, proximity sensors, accelerometers, orientation sensors and output components such as a display device, a speaker, and or a printer port. Other I/O components 1623 can be used as well.

Clock 1625 illustratively comprises a real time clock component that outputs a time and date. It can also provide timing functions for processor 1617.

Illustratively, location system 1627 includes a component that outputs a current geographical location of device 1616. This can include, for instance, a global positioning system (GPS) receiver, a LORAN system, a dead reckoning system, a cellular triangulation system, or other positioning system. It can also include, for example, mapping software or navigation software that generates desired maps, navigation routes and other geographic functions.

Memory 1621 stores operating system 1629, network settings 1631, applications 1633, application configuration settings 1635, data store 1637, communication drivers 1639, and communication configuration settings 1641. Memory 1621 can include all types of tangible volatile and non-volatile computer-readable memory devices. It can also include computer storage media (described below). Memory 1621 stores computer readable instructions that, when executed by processor 1617, cause the processor to perform computer-implemented steps or functions according to the instructions. Processor 1617 can be activated by other components to facilitate their functionality as well.

FIG. 18 shows that the device can be a smart phone 1701. Smart phone 1771 has a touch sensitive display 1773 that displays icons or tiles or other user input mechanisms 1775. Mechanisms 1775 can be used by a user to run applications, make calls, perform data transfer operations, etc. In general, smart phone 1771 is built on a mobile operating system and offers more advanced computing capability and connectivity than a feature phone.

Note that other forms of the devices 1716 are possible.

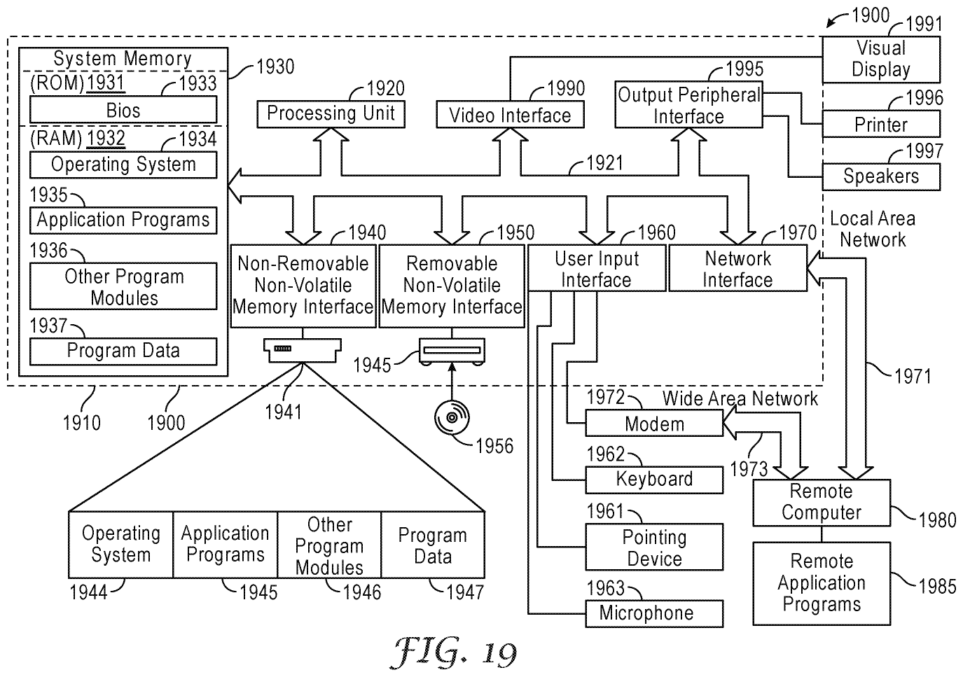

FIG. 19 is a block diagram of a computing environment that can be used in embodiments shown in previous Figures.

FIG. 19 is one example of a computing environment in which elements of systems and methods described herein, or parts of them (for example), can be deployed. With reference to FIG. 19, an example system for implementing some embodiments includes a general-purpose computing device in the form of a computer 1810. Components of computer 1810 may include, but are not limited to, a processing unit 1820 (which can comprise a processor), a system memory 1830, and a system bus 1821 that couples various system components including the system memory to the processing unit 1820. The system bus 1821 may be any of several types of bus structures including a memory bus or memory controller, a peripheral bus, and a local bus using any of a variety of bus architectures. Memory and programs described with respect to systems and methods described herein can be deployed in corresponding portions of FIG. 19.

Computer 1810 typically includes a variety of computer readable media.

Computer readable media can be any available media that can be accessed by computer 1810 and includes both volatile/nonvolatile media and removable/non-removable media. By way of example, and not limitation, computer readable media may comprise computer storage media and communication media. Computer storage media is different from, and does not include, a modulated data signal or carrier wave. It includes hardware storage media including both volatile/nonvolatile and removable/non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computer 1810. Communication media may embody computer readable instructions, data structures, program modules or other data in a transport mechanism and includes any information delivery media. The term “modulated data signal” means a signal that has one or more of its characteristics set or changed in such a manner as to encode information in the signal.

The system memory 1830 includes computer storage media in the form of volatile and/or nonvolatile memory such as read only memory (ROM) 1831 and random access memory (RAM) 1832. A basic input/output system 1833 (BIOS) containing the basic routines that help to transfer information between elements within computer 1810, such as during start-up, is typically stored in ROM 1831. RAM 1832 typically contains data and/or program modules that are immediately accessible to and/or presently being operated on by processing unit 1820. By way of example, and not limitation, FIG. 19 illustrates operating system 1834, application programs 1835, other program modules 1836, and program data 1837.

The computer 1810 may also include other removable/non-removable and volatile/nonvolatile computer storage media. By way of example only, FIG. 19 illustrates a hard disk drive 1841 that reads from or writes to non-removable, nonvolatile magnetic media, nonvolatile magnetic disk 1852, an optical disk drive 1855, and nonvolatile optical disk 1856. The hard disk drive 1841 is typically connected to the system bus 1821 through a non-removable memory interface such as interface 1840, and optical disk drive 1855 are typically connected to the system bus 1821 by a removable memory interface, such as interface 1850.

Alternatively, or in addition, the functionality described herein can be performed, at least in part, by one or more hardware logic components. For example, and without limitation, illustrative types of hardware logic components that can be used include Field-programmable Gate Arrays (FPGAs), Application-specific Integrated Circuits (e.g., ASICs), Application-specific Standard Products (e.g., ASSPs), System-on-a-chip systems (SOCs), Complex Programmable Logic Devices (CPLDs), etc.

The drives and their associated computer storage media discussed above and illustrated in FIG. 19, provide storage of computer readable instructions, data structures, program modules and other data for the computer 1810. In FIG. 19, for example, hard disk drive 1841 is illustrated as storing operating system 1844, application programs 1845, other program modules 1846, and program data 1847. Note that these components can either be the same as or different from operating system 1834, application programs 1835, other program modules 1836, and program data 1837.

A user may enter commands and information into the computer 1810 through input devices such as a keyboard 1862, a microphone 1863, and a pointing device 1861, such as a mouse, trackball or touch pad. Other input devices (not shown) may include a joystick, game pad, satellite receiver, scanner, or the like. These and other input devices are often connected to the processing unit 1820 through a user input interface 1860 that is coupled to the system bus, but may be connected by other interface and bus structures. A visual display 1891 or other type of display device is also connected to the system bus 1821 via an interface, such as a video interface 1890. In addition to the monitor, computers may also include other peripheral output devices such as speakers 1897 and printer 1896, which may be connected through an output peripheral interface 1895.

The computer 1810 is operated in a networked environment using logical connections, such as a Local Area Network (LAN) or Wide Area Network (WAN) to one or more remote computers, such as a remote computer 1880.

When used in a LAN networking environment, the computer 1810 is connected to the LAN 1271 through a network interface or adapter 1870. When used in a WAN networking environment, the computer 1810 typically includes a modem 1872 or other means for establishing communications over the WAN 1873, such as the Internet. In a networked environment, program modules may be stored in a remote memory storage device. FIG. 18 illustrates, for example, that remote application programs 1885 can reside on remote computer 1880.

A method of evaluating a surface is presented that includes imaging the surface, with an imaging system. Imaging includes providing a camera of the imaging system proximate the surface. Imaging also includes causing the imaging system and the surface to move relative to each other, such that a distance between the imaging system and the surface is substantially maintained. Imaging also includes capturing image data of the surface. The image data is captured in a near dark field mode or a dark field image mode. The method also includes analyzing the image data and detecting a topography and/or appearance of the surface. The method also includes generating an evaluation regarding the surface based on the detected topography and/or surface appearance.

The method may be implemented such that camera includes a line-scan array or an area-scan array.

The method may be implemented such that the imaging system includes a light source and wherein the near-dark field mode includes the light source and the linescan array in a first configuration with respect to the surface, and the dark field mode includes the light source and the linescan array in a second configuration.

The method may be implemented such that it includes causing the imaging system and the surface to move relative to each other a second time and capturing second image data of the surface. The second image data is captured in the near dark field mode or the dark field image mode such that the second image data is captured in a different mode than the image data.

The method may be implemented such that the second imaging system is positioned on a motive robotic arm.

The method may be implemented such that the second imaging system is mounted on an UAV.

The method may be implemented such that it includes displaying the captured image data or the detected topography on a display component.

The method may be implemented such that it includes comparing the detected topography to an expected topography.

The method may be implemented such that comparing includes detecting a change in height of the topography.

The method may be implemented such that the change in height is greater than a threshold acceptable deviation.

The method may be implemented such that the expected topography is based on a model of the surface.

The method may be implemented such that the expected topography is based on a previously detected topography.

The method may be implemented such that the surface includes curvature.

The method may be implemented such that the surface includes an edge.

The method may be implemented such that the surface includes a dispensed material.

The method may be implemented such that the imaging system includes a distance sensor that travels ahead of the camera and detects a distance between the distance sensor and the surface.

The method may be implemented such that the imaging system further includes: a controller that receives the detected distance and adjusts a position of the imaging system such that the imaging system maintains a separation distance from the surface.

The method may be implemented such that the controller stores the detected distance and such that analyzing includes analyzing the detected distance over time and reconstructing the detected topography.

A method of dispensing adhesive is presented that includes dispensing an adhesive onto a surface. The adhesive is dispensed at a speed and temperature. The method also includes imaging the surface, with an imaging system. Imaging includes: providing a camera of the imaging system proximate the surface, causing the imaging system and the surface to move relative to each other, such that a distance between the imaging system and the surface is substantially maintained, capturing image data of the surface. The image data is captured in a near dark field mode or a dark field image mode. Imaging also includes analyzing the image data and detecting a topography of the surface. The method also includes generating an evaluation regarding the surface based on the detected topography.

The method may be implemented such that the imaging step precedes the dispensing step.

The method may be implemented such that the speed is adjusted based on the detected topography.

The method may be implemented such that the imaging step follows the dispensing step.

The method may be implemented such that the topography is indicative of the dispensed adhesive.

The method may be implemented such that it includes generating a quality indication of the dispensed adhesive, wherein the quality indication includes an indication of a gap in dispensed adhesive or an indication of too much dispensed adhesive.

The method may be implemented such that it includes adjusting the speed based on the quality indication.

A surface evaluation system is presented that includes an image capturing system that captures an image of a surface. The image capturing system includes: a light source, an image capturing device configured to capture a near dark field or dark field image of the surface, and a movement mechanism configured to move the image capturing device with respect to the curved surface. The movement mechanism maintains a substantially fixed distance between the image capturing system and the surface while the image capturing device moves with respect to the surface. The system also includes a surface evaluator that receives the captured image and, based on the captured image, generates a surface quality indication. The system also includes a process parameter adjuster that adjusts a process parameter based on the surface quality indication.

The system may be implemented such that it includes a view generator that generates a view of the surface based on the images captured by the image capturing device, and a display component that presents the view.

The system may be implemented such that the surface quality indication includes a detected indentation.

The system may be implemented such that it includes a dent evaluator that provides a localized position of the dent and an indication of dent severity.

The system may be implemented such that the surface quality indication includes a detected scratch.

The system may be implemented such that it includes a scratch evaluator that provides a localized position of the scratch and an indication of scratch severity.

The system may be implemented such that the surface quality indication indicates an air bubble. The process parameter is a location of the air bubble. The system also includes a repair command generator that generates a repair command including the location of the air bubble.

The system may be implemented such that the surface quality indication indicates an amount of material added or removed from the surface, and wherein the process parameter is a location of too much or too little material.

The system may be implemented such that it includes a storage component configured to store the surface quality indication.

The system may be implemented such that it includes a path generator that receives the surface indication, wherein the surface indication is an indication of a curved surface and, based on the surface indication, the path generator generates a path for the movement mechanism that maintains a relative position of the image capturing device, the light source and the curved surface with respect to each other.

The system may be implemented such that the image capturing device, the surface and the light source form a right angle at a point on the surface being imaged.

The system may be implemented such that the surface indication includes a topography generated based on sensor information from a distance sensor array.

The system may be implemented such that the distance sensor array is coupled to the movement mechanism, and moves ahead of the image capturing device, with respect to the curved surface, and wherein the path generator generates the path and provides the path to the movement mechanism in situ.

The system may be implemented such that the image capturing device is a linescan array.

The system may be implemented such that the image capturing device is a 3D camera.

The system may be implemented such that it includes a lens between the image capturing device and the light source.

The system may be implemented such that it includes a knife edge between the image capturing device and the light source.

The system may be implemented such that the surface is a curved surface and wherein maintaining the distance includes adjusting a position of the imaging system to follow a curvature of the curved surface.

A robotic surface inspection system is presented that includes an imaging system, configured to capture an indication of a surface. The imaging system includes a light source, a knife edge positioned in front of the light source, and an image capturing device positioned such that light from the light source passes in front of the knife edge, reflects off the surface to the image capturing device. A position of the light source and the image capturing device are fixed with respect to each other during an imaging operation. The system also includes a movement mechanism that moves the imaging system with respect to the surface during the imaging operation so that a fixed distance and orientation is maintained between the surface and the imaging system is maintained. The system also includes a surface topography system that includes a distance sensor array that moves with respect to the surface, a topography generator that generates a topography based on sensor signals from the distance sensor array, and a controller that generates movement commands to the motive robotic arm that maintains a relative position of the imaging system with respect to a surface being imaged as the imaging system and the surface are moved with respect to each other. The controller generates the movement commands based on the generated topography.

The system may be implemented such that the surface is stationary and the imaging system moves with respect to the surface.

The system may be implemented such that the imaging system is stationary and wherein the surface moves with respect to the imaging system.

The system may be implemented such that the orientation includes a right angle formed between the image capturing device, the surface, and the light source.

The system may be implemented such that, in a first movement sequence, the distance sensor array captures topography information and such that, in a second movement sequence, the imaging system captures image information.

The system may be implemented such that the surface topography system and the imaging system are both active during a movement sequence. The topography generator generates the topography in-situ. The controller generates the movement commands in-situ based on received topography information from the topography generator in substantially real-time.

The system may be implemented such that it also includes a surface evaluator that provides a surface quality indication based on the image.

The system may be implemented such that it includes a display component that displays the image.

The system may be implemented such that it includes a storage component that stores the image.

The system may be implemented such that the surface is a curved surface.

The system may be implemented such that the curved surface includes curvature in two directions.

A method of generating a surface topography that includes moving a line scan array imaging system with respect to a surface. A distance between the line scan array imaging system and the surface is maintained. The method also includes imaging the surface, using the line scan array imaging system, to produce an image of the surface. The imaging system moves with respect to the surface along an imaging path. The imaging path maintains a substantially constant distance between the line scan array imaging system and the surface. The method also includes detecting, using a distance sensor associated with the line scan array imaging system, a distance change along the imaging path. The method also includes processing the distance change to generate the surface topography.

The method may be implemented such that the surface is a curved surface, and further includes: based on the detected distance change, generating a position adjustment command to adjust a position or orientation of the line scan array imaging system such that the constant distance is maintained.

The method may be implemented such that the line scan array imaging system is coupled to the distance sensor.

The method may be implemented such that the position adjustment command is generated by a controller. The line scan array imaging system is mounted to a movement mechanism, and wherein the movement mechanism executes the position adjustment mechanism.

The method may be implemented such that the movement mechanism adjusts an orientation of the line scan array imaging system to maintain an angle with respect to the curved surface.

The method may be implemented such that the movement mechanism adjusts a height of the line scan array imaging system.

The method may be implemented such that the imaging is a first imaging, and the system imaging the surface a second time.

The method may be implemented such that it includes conducting a surface processing operation between the first and second imaging.

The method may be implemented such that the imaging path is a first imaging path, the second imaging follows a second imaging path, the second imaging path is different from the first imaging path.

The method may be implemented such that the image is a first image. The second imaging produces a second image. An area of the surface is visible in both the first and second images.

The method may be implemented such that the image is a first image. The second imaging produces a second image. The first image includes a first surface area, the second image includes a second surface area. The first and second surface areas do not overlap.

The method may be implemented such that the imaging system is in a dark field configuration.

The method may be implemented such that the imaging system is in a near dark field configuration.

The method may be implemented such that the image or processed image is communicated to a display component which displays the image or processed image.

The method may be implemented such that the image or processed image is communicated to a storage component which stores the image or processed image in a retrievable form.

The method may be implemented such that the imaging system is mounted on a robotic arm, and wherein the imaging system is moved along the imaging path by the robotic arm.

The method may be implemented such that the imaging system is mounted to a UAV.

The method may be implemented such that the imaging path is generated by a controller based on a retrieved topography of the curved surface.

The method may be implemented such that the topography is provided to the controller from a distance sensor array that detects the topography as the distance sensor array travels over the curved surface.

The method may be implemented such that the distance sensor array is mounted to the robotic arm, such that the distance sensor array travels ahead of the imaging system, and wherein the controller generates the imaging path in situ based on incoming sensor signals from the distance sensor array.

The method may be implemented such that the imaging path includes the robot arm changing a relative position of the imaging system with respect to the curved surface as the imaging path is executed.

The method may be implemented such that the imaging path includes the robot arm changing a relative orientation of the imaging system with respect to the robot arm as the imaging path is executed.

The method may be implemented such that changing the relative orientation of the imaging system with respect to the robot arm maintains a relative orientation of the imaging system with respect to the curved surface as the imaging path is executed.

The method may be implemented such that the distance sensor array is mounted to a second robot arm.

The method may be implemented such that the distance sensor array is mounted to the robot arm, and wherein, in a first pass over the curved surface, the distance sensor array captures detects the topography and, in a second pass, the imaging system images the curved surface.

### EXAMPLES

### Example 1: Defect Repair Quantification

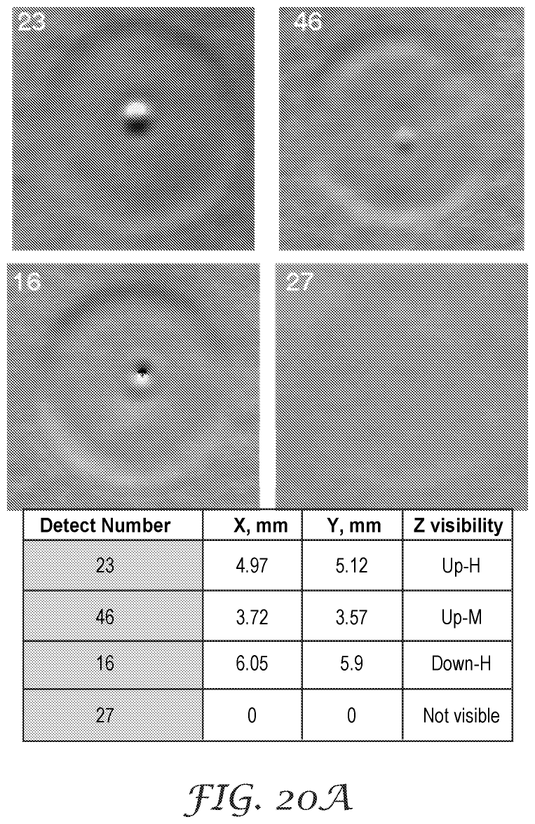

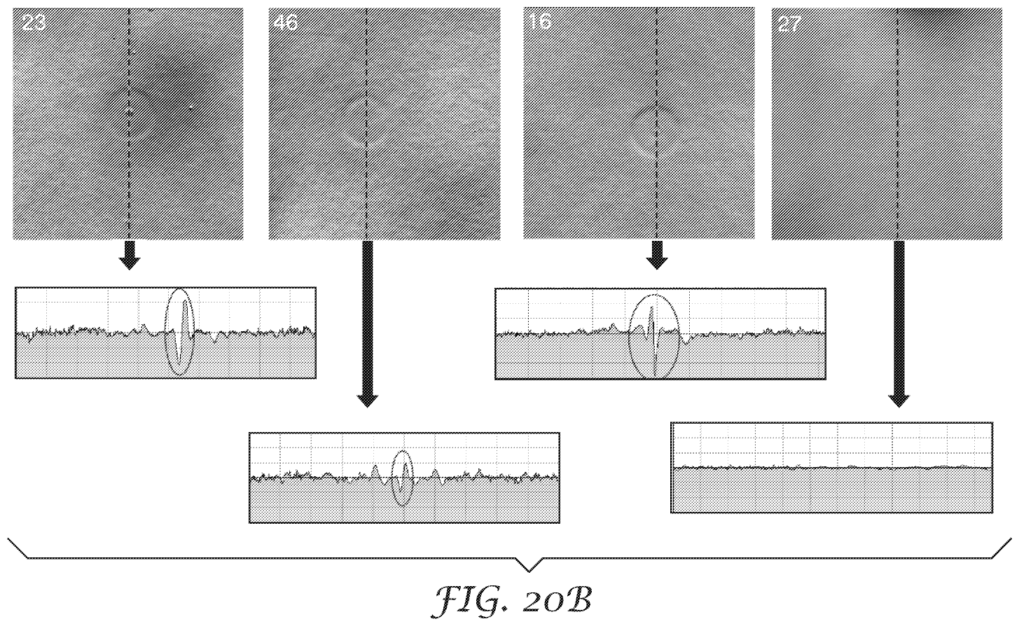

FIG. 20A illustrates images and quantification for four defect areas, taken post-repair. As illustrated, three of the defects (A, B, and D) all have visible defects, with a height characterization based on the captured images. Defect C was sufficiently removed to not be visible. As shown here, the size of the defects, in x and y directions, is measurable while the height of the defects (in z direction) can be only qualitatively evaluated. This might be useful during a post inspection process once the system only needs to decide the pass or failure of the repair. In addition, as shown in FIG. 20B, the light intensity profile taken from the defects (a′) is proportional to the height of the defects (a). In FIG. 20B, all defects shown in FIG. 20A have been characterized using a 3D non-contact laser profilometer to measure the defect's height. It shows that once the correlation between a and a′ is known (for example by a calibration procedure) the linescan array method may be used for defect's height estimation.

FIG. 20B illustrates further characterization of the defects.

1. A method of evaluating a surface, the method comprising:
imaging the surface, with an imaging system, wherein imaging comprises:
providing a camera of the imaging system proximate the surface;
causing the imaging system and the surface to move relative to each other, such that a distance between the imaging system and the surface is substantially maintained; and
capturing image data of the surface, wherein the image data is captured in a near dark field mode or a dark field image mode; and
analyzing the image data and detecting a topography and/or appearance of the surface; and
generating an evaluation regarding the surface based on the detected topography and/or surface appearance.

2. The method of claim 1, wherein camera comprises a line-scan array or an area-scan array.

3. The method of claim 2, wherein the imaging system comprises a light source and wherein the near-dark field mode comprises the light source and the linescan array in a first configuration with respect to the surface, and the dark field mode comprises the light source and the linescan array in a second configuration.

4. The method of claim 1, and further comprising:
causing the imaging system and the surface to move relative to each other a second time; and
capturing second image data of the surface, wherein the second image data is captured in the near dark field mode or the dark field image mode such that the second image data is captured in a different mode than the image data.

5. The method of claim 1, and further comprising:
comparing the detected topography to an expected topography.

6. The method of claim 5, wherein comparing comprises detecting a change in height of the topography.

7. The method of claim 1, wherein the imaging system comprises:
a distance sensor that travels ahead of the camera and detects a distance between the distance sensor and the surface.

8. The method of claim 7, wherein the imaging system further comprises:
a controller that receives the detected distance and adjusts a position of the imaging system such that the imaging system maintains a separation distance from the surface; and wherein the controller stores the detected distance and wherein analyzing comprises analyzing the detected distance over time and reconstructing the detected topography.

9. A method of dispensing adhesive, the method comprising:
dispensing an adhesive onto a surface, wherein the adhesive is dispensed at a speed and temperature;
imaging the surface, with an imaging system, wherein imaging comprises:
providing a camera of the imaging system proximate the surface;
causing the imaging system and the surface to move relative to each other, such that a distance between the imaging system and the surface is substantially maintained; and
capturing image data of the surface, wherein the image data is captured in a near dark field mode or a dark field image mode; and
analyzing the image data and detecting a topography of the surface; and
generating an evaluation regarding the surface based on the detected topography.

10. The method of claim 9, wherein the topography is indicative of the dispensed adhesive.

11. The method of claim 10 further comprising:
generating a quality indication of the dispensed adhesive, wherein the quality indication comprises an indication of a gap in dispensed adhesive or an indication of too much dispensed adhesive.

12. The method of claim 11 further comprising:
adjusting the speed based on the quality indication.

13. A surface evaluation system comprising:
an image capturing system that captures an image of a surface, wherein the image capturing system comprises:
a light source;
an image capturing device configured to capture a near dark field or dark field image of the surface; and
a movement mechanism configured to move the image capturing device with respect to the curved surface, wherein the movement mechanism maintains a substantially fixed distance between the image capturing system and the surface while the image capturing device moves with respect to the surface;
a surface evaluator that receives the captured image and, based on the captured image, generates a surface quality indication; and
a process parameter adjuster that adjusts a process parameter based on the surface quality indication.

14. The system of claim 13, wherein the surface quality indication indicates an air bubble, and wherein the process parameter is a location of the air bubble; and
a repair command generator that generates a repair command comprising the location of the air bubble.

15. The system of claim 13, wherein the surface quality indication indicates an amount of material added or removed from the surface, and wherein the process parameter is a location of too much or too little material.

16. The system of claim 13, and further comprising:
a path generator that receives the surface indication, wherein the surface indication is an indication of a curved surface and, based on the surface indication, the path generator generates a path for the movement mechanism that maintains a relative position of the image capturing device, the light source and the curved surface with respect to each other.

17. The system of claim 16, wherein the image capturing device, the surface and the light source form a right angle at a point on the surface being imaged.

18. The system of claim 13, wherein the image capturing device is a linescan array or a 3D camera.

19. The system of claim 13, and further comprising a lens between the image capturing device and the light source.

20. The system of claim 13, and further comprising a knife edge between the image capturing device and the light source.

21. The system of claim 13, wherein the surface is a curved surface and wherein maintaining the distance comprises adjusting a position of the imaging system to follow a curvature of the curved surface.

22. A robotic surface inspection system comprising:
an imaging system, configured to capture an indication of a surface, the imaging system comprising:
a light source;
a knife edge positioned in front of the light source;
an image capturing device positioned such that light from the light source passes in front of the knife edge, reflects off the surface to the image capturing device;
wherein a position of the light source and the image capturing device are fixed with respect to each other during an imaging operation; and
a movement mechanism that moves the imaging system with respect to the surface during the imaging operation so that a fixed distance and orientation is maintained between the surface and the imaging system is maintained;
a surface topography system comprising:
a distance sensor array that moves with respect to the surface; and
a topography generator that generates a topography based on sensor signals from the distance sensor array; and
a controller that generates movement commands to the motive robotic arm that maintains a relative position of the imaging system with respect to a surface being imaged as the imaging system and the surface are moved with respect to each other, and wherein the controller generates the movement commands based on the generated topography.

23. The system of claim 22, wherein the orientation comprises a right angle formed between the image capturing device, the surface, and the light source.

24. The system of claim 22, wherein the surface topography system and the imaging system are both active during a movement sequence, wherein the topography generator generates the topography in-situ, and wherein the controller generates the movement commands in-situ based on received topography information from the topography generator in substantially real-time.

25. The system of claim 22, wherein the surface is a curved surface.

26. A method of generating a surface topography, the method comprising:
moving a line scan array imaging system with respect to a surface, and wherein a distance between the line scan array imaging system and the surface is maintained;
imaging the surface, using the line scan array imaging system, to produce an image of the surface, wherein the imaging system moves with respect to the surface along an imaging path, and wherein the imaging path maintains a substantially constant distance between the line scan array imaging system and the surface;
detecting, using a distance sensor associated with the line scan array imaging system, a distance change along the imaging path; and
processing the distance change to generate the surface topography.

27. The method of claim 26, wherein the surface is a curved surface, and further comprising:
based on the detected distance change, generating a position adjustment command to adjust a position or orientation of the line scan array imaging system such that the constant distance is maintained.

28. The method of claim 26, and further comprising:
wherein the imaging is a first imaging, and
imaging the surface a second time.

29. The method of claim 28, and further comprising:
conducting a surface processing operation between the first and second imaging.

30. The method of claim 28, wherein the imaging path is a first imaging path, the second imaging follows a second imaging path, and wherein the second imaging path is different from the first imaging path.

31. The method of claim 28, wherein the image is a first image, wherein the second imaging produces a second image, and wherein an area of the surface is visible in both the first and second images.

32. The method of claim 28, wherein the image is a first image, wherein the second imaging produces a second image, and wherein the first image comprises a first surface area, the second image comprises a second surface area, and wherein the first and second surface areas do not overlap.

33. The method of claim 26, wherein the imaging system is in a dark field configuration.

34. The method of claim 26, wherein the imaging system is in a near dark field configuration.

35. The method of claim 26, wherein the imaging system is mounted on a robotic arm, and wherein the imaging system is moved along the imaging path by the robotic arm.

36. The method of claim 26, wherein the imaging system is mounted on an unmanned aerial vehicle.
